SEQUENCE LISTING (3 sequences)

SEQ ID NO: 1 - protein, 15 aa, Synthetic construct
RDFQSFRKMWPFFAM

SEQ ID NO: 2 - DNA, 45 bp, Synthetic construct
  source 1..45: mol_type = other DNA
cgcgactttcaaagtttccgtaagatgtggccgttttttgcaatg

SEQ ID NO: 3 - protein, 15 aa, Synthetic construct
  MOD_RES 1: note = Pbf (2,2,4,6,7-pentamethyl-2,3-dihydrobenzofuran-5-sulfonyl)
  MOD_RES 2: note = OtBu (t-butyl ester)
  MOD_RES 4: note = Trt (trityl)
  MOD_RES 5: note = tBu (tert-butyl)
  MOD_RES 7: note = Pbf (2,2,4,6,7-pentamethyl-2,3-dihydrobenzofuran-5-sulfonyl)
  MOD_RES 8: note = Boc (tert-butyloxycarbonyl)
  MOD_RES 10: note = Boc (tert-butyloxycarbonyl)
RDFQSFRKMWPFFAM

(19) United States
(12) Patent Application Publication
(10) Pub. No.: US 2025/0049930 A1
(43) Pub. Date: Feb. 13, 2025

(54) PROTAC MOLECULES TARGETING FOXP3 AND USES THEREOF

(71) Applicant: CEDARS-SINAI MEDICAL CENTER, Los Angeles, CA (US)

(72) Inventors: Hyung L. KIM, Los Angeles, CA (US); Yanping WANG, Culver City, CA (US); Shengchen SU, Culver City, CA (US)

(73) Assignee: CEDARS-SINAI MEDICAL CENTER, Los Angeles, CA (US)

(21) Appl. No.: 18/721,263

(22) PCT Filed: Jan. 5, 2023
(86) PCT No.: PCT/US2023/060138

§ 371 Date: Jun. 18, 2024

Related U.S. Application Data
(60) Provisional application No. 63/297,140, filed on Jan. 6, 2022.

(51) Int. Cl. A61K 47/55; A61K 31/454; A61K 39/395; A61K 47/54; A61K 47/64; A61P 35/00

(52) U.S. Cl. CPC A61K 47/55; A61K 31/454; A61K 39/3955; A61K 47/545; A61K 47/64; A61P 35/00

(57) ABSTRACT

A bi-functional, proteolysis-targeting chimera (PROTAC) agent is provided for the modulation of transcriptional factor FOXP3 and associated regulatory T cells. The agent contains an inhibitory peptide of FOXP3 covalently bound through a linker to a ubiquitin ligase binding moiety. The agent binds FOXP3 and recruits an E3 ubiquitin ligase, and thereby bringing FOXP3 in proximity to the E3 ubiquitin ligase for ubiquitination and degradation of the FOXP3. Methods of preparation and use of the agent are also provided.

### CROSS-REFERENCE TO RELATED APPLICATIONS

This application includes a claim of priority under 35 U.S.C. § 119(e) to U.S. provisional patent application No. 63/297,140, filed Jan. 6, 2022, the entirety of which is hereby incorporated by reference.

### REFERENCE TO SEQUENCE LISTING

This application contains a sequence listing submitted as an electronic xml file named, “Sequence_Listing_065472-000877WOPT” created on Dec. 28, 2022 and having a size in bytes of 6,277 bytes. The information contained in this electronic file is hereby incorporated by reference in its entirety.

### FIELD OF INVENTION

This invention relates to inhibitors of regulatory T cells and uses with immune-oncology therapies.

### BACKGROUND

Most of currently approved immuno-oncology drugs are only effective in less than 50% of patients, and the treatment effect is also short-lived. Immuno-oncology drugs can increase regulatory T cells, which is a natural response to the immune stimulation.

Therefore, it is an objective of the present invention to provide a therapy that can inhibit these regulatory T cells.

It is another objective of the present invention to use a therapy that can inhibit regulatory T cells in addition to an immuno-oncology drug to enhance the effectiveness against cancers.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The present disclosure provides a compound of chemical structure E3LBM-L-FOXP3BP, wherein the E3LBM is a ubiquitin ligase binding moiety; the L is a bond or a chemical linker that is chemically linked to the E3LBM and the PFOX3M; and the FOXP3BP is a peptide capable of binding to Forkhead box protein P3 (FOXP3), wherein upon binding of the FOXP3 to the compound, the FOXP3 is ubiquitinated by a ubiquitin ligase; or a pharmaceutically acceptable salt, enantiomer, stereoisomer, solvate, polymorph, or prodrug thereof.

According to some embodiments, the FOXP3BP is a polypeptide including an amino acid sequence RDFQSFRKMWPFFAM (SEQ ID NO:1) or a variant thereof including an amino acid sequence that has at least 99%, 98%, 97%, 96%, 95%, 90%, 80%, or 60% sequence identity to SEQ ID NO:1.

According to some embodiments, the FOXP3BP is a polypeptide consisting of an amino acid sequence of SEQ ID NO:1.

According to some embodiments, the FOXP3BP is a polypeptide variant of SEQ ID NO:1, wherein one or more residues of SEQ ID NO:1 at positions 2, 3, 5, and/or 11 are substituted with L-alanine or a D-amino acid.

According to some embodiments, the E3LBM is selected from the group consisting of pomalidomide, thalidomide, and lenalidominde.

According to some embodiments, E3LBM is a small molecule moiety that binds a ubiquitin ligase selected from the group consisting of cereblon, XIAP, VHL, and MDM2.

According to some embodiments, the L is a polyethyleneglycol optionally substituted with aryl or phenyl, having from 1 to 10 ethylene glycol units.

The present disclosure provides a pharmaceutical composition including one or more compounds of the above-described compounds, further including a pharmaceutically acceptable carrier, additive or excipient, and optionally further including an additional bioactive agent.

According to some embodiments, the pharmaceutical composition includes the additional bioactive agent, which is selected from the group consisting of an mTOR inhibitor, a PD-1 inhibitor, a PD-L1 inhibitor, an immune checkpoint inhibitor, immunomodulating drugs, and a combination thereof. According to some embodiments, the mTOR inhibitor comprises temsirolimus. According to some embodiments, the PD-1 inhibitor is an anti-PD-1 antibody.

According to some embodiments, the PD-1 inhibitor comprises Pembrolizumab (Keytruda), Nivolumab (Opdivo), Dostarlimab, and Cemiplimab (Libtayo). According to some embodiments, the PD-L1 inhibitor comprises Atezolizumab (Tecentriq), Avelumab (Bavencio), and Durvalumab (Imfinzi). According to some embodiments, the immune checkpoint inhibitor comprises LAG-3 inhibitors comprising Relatlimab. According to some embodiments, the immunomodulating drugs comprises Thalidomide (Thalomid), lenalidomide (Revlimid), and pomalidomide (Pomalyst).

The present disclosure provides a method of inhibiting growth of a tumor in a subject, or treating the subject having a cancer. In various embodiments, the method includes administering to the subject a therapeutically effective amount of any one of the above-described compounds.

According to some embodiments, in the method, the E3LBM of the compound is derived from pomalidomide, the L of the compound is a polyethyleneglycol, and the FOXP3BP of the compound is a polypeptide having an amino acid sequence of SEQ ID NO:1 or a variant thereof.

According to some embodiments, the method further includes administering to the subject a therapeutically effective amount of an mTOR inhibitor.

According to some embodiments, the method further includes administering to the subject a therapeutically effective amount of a PD-1 inhibitor, a PD-L1 inhibitor, an immune checkpoint inhibitor, immunomodulating drugs, or a combination thereof.

According to some embodiments, in the method, the administration includes administering for about 2 weeks, 3 weeks, 4 weeks, or more, and the tumor in the subject is smaller in size, or the growth of the tumor as a percentage of initial size prior to the administration is smaller in the subject, compared to a non-treated subject having the cancer.

According to some embodiments, in the method, the administration includes administering for about 2 weeks, 3 weeks, 4 weeks, or more, and the tumor in the subject is smaller in size, or the growth of the tumor as a percentage of initial size prior to the administration is smaller in the subject, compared to a subject having the cancer and treated without a combination of the compound and the mTOR inhibitor.

According to some embodiments, in the method, the administration includes administering for about 2 weeks, 3 weeks, 4 weeks, or more, and the tumor in the subject is smaller in size, or the growth of the tumor as a percentage of initial size prior to the administration is smaller in the subject, compared to a subject having the cancer and treated without a combination of the compound and the PD-1 inhibitor, a PD-L1 inhibitor, an immune checkpoint inhibitor, immunomodulating drugs, or a combination thereof.

The present disclosure provides a method of improving an anti-tumor effect of an immuno-oncology drug in a subject. In various embodiments, the method includes administering to the subject a therapeutically effective amount of any one of the above-described compounds, wherein the subject has been treated or is in need of a treatment with the immuno-oncology drug.

According to some embodiments, in the method, the immuno-oncology drug includes an mTOR inhibitor, a PD-1 inhibitor, or both.

According to some embodiments, in the method, the therapeutically effective amount of the compound is administered for about 2 weeks, 3 weeks, 4 weeks, or more.

According to some embodiments, in the method, the compound is effective for reducing a number of CD4+ T cells that express the FOXP3 transcription factor in the subject.

### BRIEF DESCRIPTION OF THE FIGURES

FIGS. 1A and 1B depict exemplary reaction schemes in preparing a conjugate between pomalidomide and RDFQSFRKMWPFFAM (SEQ ID NO:1), a FOXP3 binding peptide termed “P60”, via a PEGylated crosslinker: PEG containing 4 repeating units of oxyethylene (—O—CH2-CH2-). FIG. 1A depicts a reaction scheme to prepare pomalidomide-PEG₄-CO₂H, synonym: 1-((2-(2,6-dioxopiperidin-3-yl)-1,3-dioxoisoindolin-4-yl)amino)-3,6,9,12-tetraoxapentadecan-15-oic acid. Specifically, 2-(2,6-dioxopiperidin-3-yl)-4-fluoroisoindoline-1,3-dione was reacted with amino-PEG4-t-butyl ester in exemplary co-solvents N, N-Disopropylethylamine and N-methyl-2-pyrrolidone from room temperature to 85° C.; followed by trifluoroacetic acid (TFA) treatment to remove the t-butyl protecting group. Alternatively, pomalidomide-PEG₄-CO₂H is available via suppliers such as SigmaAldrich. The pendent carboxylic acid provides for reactivity with an amine on a peptide. FIG. 1B depicts solid phase peptide synthesis (SPPS) of a protected version of the P60 peptide on a Fmoc-Met-Wang resin (SEQ ID NO:3), followed by conjugation with pomalidomide-PEG₄-CO₂H and removal of the protecting groups from the conjugated peptide. Referring to SEQ ID NO:3 shown in FIG. 1B, protecting groups on the peptide include Boc (tert-butyloxycarbonyl), Pbf (2,2,4,6,7-pentamethyl-2,3-dihydrobenzofuran-5-sulfonyl), tBu (tert-butyl), Trt (trityl), and OtBu (t-butyl ester), which are removal by TFA, 90% TFA, 90% TFA, 90% TFA, 50% TFA, respectively.

FIGS. 2A-2E depict that a proteolysis-targeting chimera (PROTAC) targeting FoxP3 (PF) degraded FoxP3 in vitro and in vivo. P-FoxP3 forms a ternary complex with cereblon and FoxP3. Surface plasmon resonance (SPR) assay was used to measure the equilibrium dissociation constant (KD) for the ternary complex consisting of cereblon, P-FoxP3 and FoxP3 (FIG. 2A). SPR assay was used to measure the KD for the binary complex consisting of cereblon and P-FoxP3 (FIG. 2B). Mouse splenocytes were treated in vitro with varying PF concentrations and percent of CD4+ cells staining for intracellular FoxP3 was assessed by flow cytometry. (FIG. 2C). Mice received 0.1 mmole of PF intraperitoneally (i.p.) (FIGS. 2D and 2E). 72 hours later, FoxP3 positive CD4+ cells in peripheral blood and spleen were assessed by flow cytometry (FIG. 2D). FoxP3 positive CD4+ cells in peripheral blood were assessed by flow cytometry at various timepoints following PF administration (FIG. 2E). Representative results from duplicate experiments are shown.

FIGS. 3A-3D depict that PF decreased the immunosuppressive function of Tregs in vitro and decreased tumor growth in mice. To treat Tregs, CD4+ splenocytes were isolated and treated with PF or various controls for 72 hours. To assess the immunosuppressive effects of these cells, they were washed and co-cultured with effector cells, which were stimulated with antibodies to CD3 and CD28 in vitro (FIGS. 3A-3C). CD8 proliferation was measured using a CFSE dilution assay. To assess CD8+ effector cells, CD4 negative splenocytes were CFSE labeled and CFSE dilution was measured in CD8+ cells by flow cytometry (FIG. 3A). In the same experiment, CD8+ lymphocyte activation was assessed by measuring TNFα (FIG. 3B). Also, CD4+ lymphocyte activation was assessed by measuring TNFα (FIG. 3C). Mice tumors were established by injection RENCA cells subcutaneously in the flank of Balb/C mice (5-7 mice per group) (FIG. 3D). 10 days later, mice were treated i.p., twice a week throughout the experiment, with the indicated drugs and tumor growth was monitored. Pomalidomide conjugated to (PEG)4 (PROTAC-control; PC) and P60 served as controls. Representative results from duplicate experiments are shown.

FIGS. 4A-4D depict that PF enhanced the antitumor immunity provided by αPD1 or mTOR inhibition in mice. Mice tumors were established by injecting RENCA-CA9 cells subcutaneously in the flank (day 0) of Balb/C mice (6-8 mice per group). PF was administered i.p., twice a week, throughout the experiment, starting on day 10 (FIGS. 4A and 4C). αPD1 was administered i.p., twice a week, throughout the experiment, starting on days 5 (FIG. 4A). Splenocytes were harvested, and restimulated ex vivo with mouse CA9 peptide. The splenocytes were assessed by flow cytometry for Granzyme B, IFNγ or TNFα after gating on CD4 or CD8 (FIG. 4B). In a similar mouse tumor growth experiment, temsirolimus was administered i.p. on day 10, every other day, for 14 days (FIG. 4C). Similar to FIG. 4B, splenocytes were harvested, restimulated and analyzed (FIG. 4D). Representative results are shown for experiments conducted at least twice. Abbreviations—Proteolysis-targeting chimera (PROTAC) targeting FoxP3 (PF); temsirolimus (Tem).

FIGS. 5A-5E depict that in combination therapy with PF plus αPD1 or mTOR inhibition, antitumor immunity was CD8+ lymphocyte dependent. Mice tumor were established by injection RENCA cell subcutaneously in the flank of Balb/C mice (6-8 mice per group). 10 days later, mice were treated with PF twice per week. CD8 lymphocytes were depleted with αCD8 administered i.p. 2 days after injection of tumor cells. Tumor growth was monitored, Mice in indicated groups received i.p. αPD1, twice a week, starting day 5 (FIG. 5A). Mice in indicated groups received i.p. temsirolimus every other day for 14 days, starting on days 10 (FIG. 5B). To determine if immunity is lymphocyte-dependent, RENCA-bearing mice were treated with 4 doses of PF and 7 doses of temsirolimus distributed over 14 days. Spleen and lymph nodes were harvested from these mice, and CD8+ cells were isolated and cultured in vitro with tumor lysate-pulsed DC and IL-2 for 3 days. 2×10⁶ CD8+ cells were adoptively transferred by tail vein injection into recipient mice, which were challenged with intravenous (i.v.) RENCA cells. Lung tumors were examined 30 days later (FIG. 5C). Lung weights (FIG. 5D) and number of tumors visible on the lung surface (FIG. 5E) are shown. Histograms are labeled with the treatment provided to the donor mice. Abbreviation—Proteolysis-targeting chimera (PROTAC) targeting FoxP3 (PF).

FIGS. 6A-6C depict that PF enhances immune activation and CD8+ memory lymphocyte formation. Mice tumors were established by injecting RENCA-CA9 cell subcutaneously in the flank of Balb/C mice (6-8 mice per group), which were treated with the indicated drugs. PF (2×/week, throughout experiment) and temsirolimus (3×/week for 2 wks) treatment administered i.p., starting 10 days after RENCA injection (FIG. 6A). Spleen and lymph nodes were harvested on day 28 and restimulated ex vivo with CA9 peptide and assessed by flow cytometry (FIGS. 6B and 6C). CD8+ lymphocytes treated with PF had increased activation as measured by IFNγ or TNFα (FIG. 6B). PF-treated CD8+ lymphocytes displayed a memory phenotype with increase in CD62L, CD44 and Eomes expression (FIG. 6C). Representative results from duplicate experiments are shown. Abbreviations—Proteolysis-targeting chimera (PROTAC) targeting FoxP3 (PF); Pomalidomide conjugated to (PEG)4 (PC); Temsirolimus (Tem).

FIGS. 7A-7C depict that PF decreases FoxP3 and the immunosuppressive function of human CD4+ cells. Human peripheral blood mononuclear cells (PBMC) were treated in vitro with PF (10 μM) or various control drugs (10 μM) for 3 days (FIG. 7A). PMA 10 ng/ml and Ionomycin 2 μg/ml were added during PF treatment to induce FoxP3 (FIG. 7A). Resulting cells were gated on CD4 and assessed for FoxP3 expression by flow cytometry. To treat Tregs, CD4+ PBMC were isolated and treated with PF or various controls for 72 hours. To assess the immunosuppressive effects of these cells, they were washed and co-cultured with CD4 negative PBMC (effector cells), which were stimulated with antibodies to CD3 and CD28 ex vivo (FIGS. 7B and 7C). CD8 proliferation was measured using a CFSE dilution assay. To assess CD8+ effector cells, CD4 negative splenocytes were CFSE labeled and CFSE dilution was measured in CD8+ cells by flow cytometry (FIG. 7B). In the same experiment, CD8+ cells were assessed for activation by gating on CD8 and assessing IFNγ or TNFα by flow cytometry (FIG. 7C). Representative results from duplicate experiments are shown. Abbreviations—Proteolysis-targeting chimera (PROTAC) targeting FoxP3 (PF); Pomalidomide conjugated to (PEG)4 (PC).

FIG. 8A depicts that P60 alone had no effect on RENCA tumor growth (tumor volume in mm³ over days following administration). FIG. 8B depicts that PF does not alter growth of RENCA cells in vitro.

FIGS. 9A and 9B depict that P-FoxP3 degraded FoxP3 in CD4+ cells. Fresh mouse splenocytes were cultured (with IL-2 at 10 μm/ml) in vitro and treated with 1 μm P-FoxP3 or without P-FoxP3 (control) for 7 days. Flow cytometry (FIG. 9A) and summarized data (FIG. 9B) are shown.

FIGS. 10A-10C depict that P-FoxP3 inhibited tumor growth when combined with an mTOR inhibitor or an αPD-1 antibody. Balb/c mice of 5-8 weeks' old were subcutaneously injected with RENCA cells. When tumor reach approximately 40 mm³, the mice were treated intraperitoneally with PBS, P-FoxP3 (250 g/dose, 2×/week), αPD-1 antibody (200 μg/dose, 2×/week), temsirolimus (750 μg/kg/dose, daily). FIG. 10A shows tumor size in P-FoxP3 treated mice was smaller, and tumor growth slower, compared to control mice, especially around 30 days or 40 days after initial tumor cell injection. FIG. 10B shows tumor growth and size were slower and smaller in mice treated with both P-FoxP3 and αPD-1 antibody, compared to other mice in this graph (p=0.0018, P-FoxP3+αPD-1 vs P-FoxP3). FIG. 10C shows tumor growth and size were slower and smaller in mice treated with both P-FoxP3 and temsirolimus, compared to mice treated with temsirolimus alone (p<0.0001).

Exemplary embodiments are illustrated in referenced figures. It is intended that the embodiments and figures disclosed herein are to be considered illustrative rather than restrictive.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor NY, 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “compound” or “chemical compound” can include organometallic compounds, organic compounds, metals, transitional metal complexes, and small molecules. The term “small molecule” generally refers to a non-peptidic, non-oligomeric organic compound either synthesized in the laboratory or found in nature. A small molecule is typically characterized in that it contains several carbon-carbon bonds, and has a molecular weight of less than 2000 g/mol, preferably less than 1500 g/mol, although this characterization is not intended to be limiting for the purposes of the present application.

The term “prodrug” is a biologically inactive compound/medication which can be metabolized in the body to produce a drug. A prodrug is a medication or compound that, after intake, is metabolized into a pharmacologically active drug. Instead of administering a drug directly, a corresponding prodrug can be used to improve how the drug is absorbed, distributed, metabolized, and excreted.

In chemistry, a “derivative” is a compound that is derived from a similar compound by some chemical or physical process. It is also used to mean that a compound can arise from another compound, if one atom is replaced with another atom or group of atoms. A term “structural analogue” can be also used for this meaning.

The term “structural analogue” or term “analogue” describes structural and functional similarity. The analogue of an existing drug molecule is indicated to share structural and pharmacological similarities with the original compound. For example, lenalidomide and pomalidomide are among thalidomide analogs, and are believed to act in a similar fashion.

The term “linker” or “linker group” refers to a chemical moiety utilized to attach one part of a compound of interest to another compound of interest. A PROTAC linker is a crosslinker that connects two functional motifs of a PROTAC, a target protein binder and an E3 ligase recruiter. Most commonly used PROTAC linkers in the development of PROTACs are PEGs, Alkyl-Chain and Alkyl/ether. Insertion of PEG increases the water-solubility of the molecules.

Examples of PROTAC linkers include but are not limited to VHL Ligand 1,(S,R,S)-AHPC-Boc, (S,R. S)-AHPC-Acid, (S,R. S)-AHPC-PEG-Acid, (S,R.S)-AHPC-PEG-NHS Ester, (S,R. S)-AHPC-Amine, (S,R. S)-AHPC-PEG-Amine, (S,R. S)-AHPC-PEG-Azide, (S,R. S)-AHPC-PEG-Tosyl, E3 Ligase Ligand 1a, GMB-475, EGFR PROTAC, MZl, E3 Ligase Ligand 19, VH-298, LC-2, Gefitinib-Based PROTAC 3, E3 Ligase Ligand 9, Thalidomide, Thalidomide-O-Acid, Thalidomide-O-PEG-Acid, Thalidomide-O—C6-Amine HCl, Thalidomide-O-PEG-Amine, Thalidomide-O-PEG-Azide, Thalidomide-O-PEG-Propargyl, Thalidomide-O-PEG-NHS Ester, Thalidomide-O-PEG-T-Butyl Ester, Thalidomide-O-PEG-Tosyl, Thalidomide-O-Acetamido-C4-Amine, Thalidomide-O-Amido-PEG-Amine, Thalidomide-O-Amido-PEG4-Azide, Thalidomide-O-Amido-PEG4-Propargyl, Thalidomide-5-Amine, Thalidomide-5-Acid, D-Biotin-PEG-Thalidomide, D-Amino-PEG-Thalidomide, DBET1, DBET 6, BETd-260, Pomalidomide, Pomalidomide-PEG-Ph-NH2, Pomalidomide 4′-PEG-Azide, Pomalidomide-PEG-Azide, Homo-Protac Cereblon Degrader 1, P131, ARV-825, MS4078, ARV-110, THAL-SNS-032, Lenalidomide, Iberdomide, CFT7455, NVP-DKY709, CC-90009, Avadomide, and 4-Amino-2-(1-Methyl-2,6-Dioxopiperidin-3-Yl)Isoindoline. For example, these linkers are available from BroadPharm (San Diego, CA).

The terms “polypeptide,” “peptide,” and “protein” also encompass an amino acid polymer that has been modified naturally or by intervention; for example, disulfide bond formation, lactam bridge formation, glycosylation, lipidation, acetylation, acylation, amidation, phosphorylation, or other manipulation or modification, such as conjugation with a labeling component or addition of a protecting group. Also included within the definition are, for example, polypeptides containing one or more analogs of an amino acid (including, for example, amino-isobutyric acid (Aib), unnatural amino acids, etc.) and polypeptides comprising or consisting of D-amino acids, as well as other modifications known in the art. In certain embodiments, the polypeptides can occur as single chains, covalent dimers, or non-covalent associated chains. Polypeptides can also be in cyclic form. Cyclic polypeptides can be prepared, for example, by bridging free amino and free carboxyl groups. Formation of the cyclic compounds can be achieved by treatment with a dehydrating agent, with suitable protection if needed. The open chain (linear form) to cyclic form reaction can involve intramolecular-cyclization. Cyclic polypeptides can also be prepared by other methods known in the art, for example, using one or more lactam bridges, hydrogen bond surrogates (Patgiri et al. 2008), hydrocarbon staples (Schafmeister et al. 2000), triazole staples (Le Chevalier Isaad et al. 2009), or disulfide bridges (Wang et al. 2006). Bridges or staples can be spaced, for example, 3, 4, 7, or 8 amino acids apart.

The term “variant” refers to a peptide having one or more amino acid substitutions, deletions, and/or insertions compared to a reference sequence. Deletions and insertions can be internal and/or at one or more termini. Substitution can include the replacement of one or more amino acids with a similar or homologous amino acid(s) or a dissimilar amino acid(s). For example, some variants include alanine substitutions at one or more amino acid positions. Other substitutions include conservative substitutions that have little or no effect on the overall net charge, polarity, or hydrophobicity of the protein. Some variants include non-conservative substitutions that change the charge or polarity of the amino acid. Substitution can be with either the L- or the D-form of an amino acid.

The term “conservative substitution” as used herein denotes that one or more amino acids are replaced by another, biologically similar residue. Examples include substitution of amino acid residues with similar characteristics, e.g., small amino acids, acidic amino acids, polar amino acids, basic amino acids, hydrophobic amino acids, and aromatic amino acids.

In the scheme below, conservative substitutions of amino acids are grouped by physicochemical properties; I: neutral and/or hydrophilic, II: acids and amides, III: basic, IV: hydrophobic, V: aromatic, bulky amino acids.

| I | II | III | IV | V |
|---|---|---|---|---|
| A | N | H | M | F |
| S | D | R | L | Y |
| T | E | K | I | W |
| P | Q | | V | |
| G | | | C | |

In the scheme below, conservative substitutions of amino acids are grouped by physicochemical properties; VI: neutral or hydrophobic, VII: acidic, VIII: basic, IX: polar, X: aromatic.

| VI | VII | VIII | IX | X |
|---|---|---|---|---|
| A | D | H | M | F |
| L | E | R | S | Y |
| I | | K | T | W |
| V | | | N | H |
| P | | | Q | |
| G | | | C | |

Tregs are the master regulator for adaptive immunity. FoxP3 expression is necessary and sufficient to provide CD4+ lymphocytes with suppressive Treg function. However, it is difficult to use FoxP3 as a drug because it is a transcription factor and not a target for antibodies or small molecule kinase inhibitors. Yet, it is an important target in immuno-oncology based on observations in genetically engineered mice where it possible to specifically deplete Tregs. For example, in mice expressing the diphtheria toxin receptor (DTR) behind the FoxP3 promoter, diphtheria toxin (DT) decreases the growth of established tumors. It is also important to note that targeting FoxP3 for cancer therapy appears safe. Although, mice and humans born with defects in FoxP3 have severe autoimmunity, targeting FoxP3 expression in adult mice appears to have no appreciable adverse effects.

PROTACs are a class of drugs consisting of two molecules joined by a linker. One of the molecules binds a target ligand and the other molecule recruits an E3 ubiquitin ligase. PROTACs induce ubiquitylation of the target. Described herein we created a PROTAC that targets FoxP3 for degradation, using a FoxP3-binding oligopeptide designated as P60, which was identified from a phage-displayed random peptide library. Molecules like P60 bind and temporarily inhibit the function of the target protein. However, classic inhibitors can leave the target under drugged when it is cleared, allowing the target to regain function. PROTACS, on the other hand, completely degrade the target protein and are recycled for continuous degradation of remaining target proteins. Therefore, PROTACs can have a wider therapeutic window and improved potency, while maintaining target selectivity. Furthermore, PROTACs are less susceptible to drug resistance mechanisms induced by mutation or overexpression of the target protein.

Herein, we showed that PF binds to both FoxP3 and cereblon, an E3 ubiquitin ligase, resulting in degradation of FoxP3 by the ubiquitin-proteasome system. PF decreased FoxP3 level in a dose-dependent manner in vitro. In mice, PF decreased FoxP3 in both spleen and peripheral lymphocytes, an effect that lasted at least 8 days after a single PF dose. It was then important to document that PF treated Tregs had decreased immunosuppressive function. In mixed culture experiments, we showed that PF treated lymphocytes were better at stimulating CD8+ lymphocyte proliferation and activation. These observations were noted with both mouse and human lymphocytes. Consistent with this observation, PF treatment decreased tumor growth in mice, and PF was more effective at suppressing tumor growth than P60 or a PROTAC-control.

Checkpoint inhibitors are proving effective against an increasing array of cancer types. MTOR inhibitors are also approved for multiple cancers, and mTORi work at least in part by modulating immunity. Therefore, we assessed PF in combination with a PD-1 antibody (αPD1) or a mTORi in mouse models. PF enhanced the antitumor immunity associated with αPD1 or mTORi, decreasing tumor growth, enhancing antitumor CD8+ lymphocyte function and increasing formation of CD8+ central memory cells. To confirm that the antitumor activity from PF and mTORi is lymphocyte-dependent, a lymphocyte transfer study was performed in which mice never exposed to other immune modulating drugs were challenged with RENCA tumor cells intravenously. In these mice, lymphocytes from mice treated with PF and mTORi were sufficient to suppress growth of metastatic tumor deposits. This model rules out the possibility of antitumor drug activity against tumor cells since RENCA tumor cells are never directly exposed to PF or mTORi.

We show herein that targeting the immune system with a PROTAC drug can effectively inhibit tumor growth, and PROTACs can be effective for immuno-oncology targets that are difficult to target with antibody or small molecule drugs.

In conclusion, PF decreases FoxP3 expression in Tregs, reducing Treg function and generating antitumor immunity in preclinical mouse models and human mixed culture experiments, providing a rationale for clinical testing.

Regulatory T cells (Treg) possess immunosuppressive activity which prevents chronic inflammation and maintains peripheral immune tolerance, thereby protecting the host against autoimmune diseases. However, this immunoregulatory function also restrains the induction of immune responses against cancer and infectious agents. Treg are characterized by the expression of CD25 and the Treg-specific FOXP3 transcription factor, which is believed as required for their development and function.

“FOXP3BP” refers to a binding moiety, preferably a peptide, capable of binding to Forkhead box protein P3 (FOXP3). This FOXP3 binding moiety is linked to the ubiquitin ligase binding moiety preferably through a linker, such that a target protein, FOXP3, is presented in proximity to the ubiquitin ligase for ubiquitination and degradation.

Some aspects of the invention provide drugs that specifically target CD4+ FoxP3+ regulatory T cells. Some aspects of the invention provide formulations of drugs that specifically target CD4+ FoxP3+ regulatory T cells. Some aspects of the invention provide methods of using drugs that specifically target CD4+ FoxP3+ regulatory T cells to treat a cancer or tumor.

A (bifunctional) compound is provided, which has or comprises a chemical structure of E3LBM-L-FOXP3BP, wherein the E3LBM is a ubiquitin ligase binding moiety; the L is a bond or a chemical linker that is chemically linked to the E3LBM and the PFOX3M; and the FOXP3BP is a peptide capable of binding to Forkhead box protein P3 (FOXP3), wherein upon binding of the FOXP3 to the compound, the FOXP3 is ubiquitinated by a ubiquitin ligase.

In various aspects, the FOXP3BP is a polypeptide comprising an amino acid sequence set forth in, or consisting of an amino acid sequence of, RDFQSFRKMWPFFAM (SEQ ID NO:1) or a variant thereof comprising an amino acid sequence that has at least 60%, such as at least 80%, at least 85%, at least 90%, at least 95%, at least 96%, at least 97%, at least 98% or at least 99% sequence identity with the amino acid sequence set forth in SEQ ID NO:1 over the entire length of SEQ ID NO:1. The variant may comprise an amino acid sequence which has one or more, such as two, three, four, five or six amino acid substitutions compared to SEQ ID NO:1. Preferably, such amino acid substitution is a conservative substitution which means that one amino acid is replaced by another one that is similar in size and chemical properties. Such conservative amino acid substitution may thus have minor effects on the peptide structure and can thus be tolerated without compromising function. Preferably, such variant is capable of binding FOXP3 or inhibiting the activity of FoxP3. In some embodiments, the substitution is alanine. In some embodiments, amino acids at positions 2, 3, 5, 11 or a combination thereof of SEQ ID NO:1 are substituted with alanine. FoxP3 binding by P60 can be improved by these substitutions. In some embodiments, provide for a D-amino acid substitution in SEQ ID NO:1; for example, at position 2 of SEQ ID NO:1. These embodiments can augment microsomal stability while maintaining FOXP3 binding. An illustrative nucleotide sequence coding for this polypeptide having the amino acid sequence of SEQ ID NO:1 is represented by CGCGACTTTCAAAGTTTCCGTAAGATGTGGCCGTTTTTTGCAATG (SEQ ID NO:2).

In some aspects, the FOXP3BP is a polypeptide variant of native P60, containing substitution at positions 3, 5, and/or 11 (residues F, S, and P, respectively) by alanine, which exhibits slightly improved binding capacity to FOXP3 than that of P60. In some aspects, the FOXP3BP is a polypeptide variant of P60, containing substitution of aspartic at position 2 by alanine, termed P60-D2A, which exhibits an improved binding capacity to FOXP3 than that of P60. In other aspects, the FOXP3BP is a polypeptide variant of P60, wherein positions 1, 6, 7, 8, 10, 12, 13 and 15 (residues R, F, R, K, M, W, F, F and M respectively) are not replaced by alanine, as that would harm the binding to FOXP3.

In some aspects, the FOXP3BP is from native P60 or a polypeptide variant of P60, wherein the FOXP3BP has an N-terminal acetylation and/or C-terminal amidation.

In some aspects, the FOXP3BP is a P60 derived peptide, wherein one or more L-amino acids in P60 (or its variant) are substituted with D-amino acids (i.e., D-amino acid in place of L-amino acid). In further aspects, substitution with D-amino acids results in FOXP3BP that is more resistant to proteolytic degradation but retains binding capacity to FOXP3. In some aspects, the FOXP3BP is a P60 derived peptide, wherein residues in position 2, 3, 5, and/or 11 of native P60 is replaced with another D-enantiomer amino acid, such as D-alanine.

In further aspects, the FOXP3BP is a cyclic peptide derived from P60.

In some embodiments, a mixture of compounds having a chemical structure of: E3LBM-L-FOXP3BP are provided, or for use in treating a subject with cancer, wherein each compound has a different FOXP3BP sequence, based on native P60, one or more variants thereof, or both.

In various aspects, the E3LBM is capable of binding, or binds to, a ubiquitin ligase. E3 ubiquitin ligases (of which over 600 are known in humans) confer substrate specificity for ubiquitination. As described herein, a ubiquitin ligase binding moiety is a small molecule or a peptide that can bind an E3 ubiquitin ligase.

Specific E3 ubiquitin ligases include cereblon, von Hippel-Lindau (VHL); X-linked inhibitor of apoptosis (XIAP), E3A; MDM2; Anaphase-promoting complex (APC); UBR5 (EDD1); SOCS/BC-box/eloBC/CUL5/RING; LNXp80; CBX4; CBLL1; HACE1; HECTD1; HECTD2; HECTD3; HECW1; HECW2; HERC1; HERC2; HERC3; HERC4; HUWE1; ITCH; NEDD4; NEDD4L; PPIL2; PRPF19; PIAS1; PIAS2; PIAS3; PIAS4; RANBP2; RNF4; RBX1; SMURF1; SMURF2; STUB1; TOPORS; TRIP12; UBE3A; UBE3B; UBE3C; UBE4A; UBE4B; UBOX5; UBR5; WWP1; WWP2; Parkin; A20/TNFAIP3; AMFR/gp78; ARA54; beta-TrCP1/BTRC; BRCA1; CBL; CHIP/STUB1; E6; E6AP/UBE3A; F-box protein 15/FBXO15; FBXW7/Cdc4; GRAIL/RNF128; HOIP/RNF31; cIAP-1/HIAP-2; cIAP-2/HIAP-1; cIAP (pan); ITCH/AIP4; KAP1; MARCH8; Mind Bomb 1/MIB1; Mind Bomb 2/MIB2; MuRF1/TRIM63; NDFIP1; NEDD4; NleL; Parkin; RNF2; RNF4; RNF8; RNF168; RNF43; SART1; Skp2; SMURF2; TRAF-1; TRAF-2; TRAF-3; TRAF-4; TRAF-5; TRAF-6; TRIM5; TRIM21; TRIM32; UBR5; and ZNRF3.

In various implementations, the ubiquitin ligase is cereblon. Without wishing to be bound by a particular theory, cereblon is a protein that forms an E3 ubiquitin ligase complex with damaged DNA binding protein 1 (DDB1), Cullin-4A (CUL4A), and regulator of cullins 1 (ROC1); and this complex ubiquitinates a number of other proteins, resulting in increased levels of fibroblast growth factor 8 (FGF8) and fibroblast growth factor 10 (FGF10). FGF8 in turn regulates a number of developmental processes, such as limb and auditory vesicle formation. In the absence of cereblon, DDB1 forms a complex with DDB2 that functions as a DNA damage-binding protein.

Pomalidomide, thalidomide, and lenalidomide, and analogs thereof bind to cereblon. The crystal structure of cereblon with thalidomide and derivative compounds are described in US2015/0374678, which is incorporated by reference. Other small molecule compounds that bind to cereblon are disclosed as an in US2016/0058872 and US2015/0291562, which are incorporated by reference.

For other ubiquitin ligase, such as VHL, MDM2, and XIAP, their small molecular binding compounds are described in publications such as US20190175612, which is herein incorporated by reference.

In various aspects, the L in the compound is -A₁ . . . A_{q}-; wherein A₁ to A_{q} are each independently selected from the group consisting of a bond, CR^{L1}R^{L2}, O, S, S═O, S(═O)₂, NR^{L3}S(═O)₂NR^{L3}, S(═O)NR^{L3}, C(═O)NR^{L3}, NR^{L3}C(═O)NR^{L4}, NR^{L3}S(═O)₂NR^{L4}, C(═O), CR^{L1}═CR^{L2}, C≡C, SiR^{L1}R^{L2}, P(═O)R^{L1}, P(═O)OR^{L1}, NR^{L3}C(═N—CN)NR^{L4}, NR^{L3}C(═N—CN), NR^{L3}C(═C—NO₂)NR⁴, C₃₋₁₁ cycloalkyl optionally substituted with 0-6 substituents selected from the group consisting of R^{L1} and R^{L2}, C₃₋₁₁ heterocyclyl optionally substituted with 0-6 substituents selected from the group consisting of R^{L1} and R^{L2}, aryl optionally substituted with 0-6 substituents selected from the group consisting of R^{L1} and R^{L2}, and heteroaryl optionally substituted with 0-6 substituents selected from the group consisting of R^{L1} and R^{L2}, wherein: R^{L1} and R^{L2} each independently can be linked to other A groups to form a cycloalkyl or heterocyclyl moeity that can be further optionally substituted with 0-4 R^{L5} groups; R^{L1}, R^{L2}, R^{L3}, R^{L4}, and R^{L5} are each independently selected from the group consisting of H, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocyclyl, O(C₁₋₈ cycloalkyl), S(C₁₋₈ cycloalkyl), NH(C₁₋₈ cycloalkyl), N(C₁₋₈ cycloalkyl)₂, N(C₁₋₈ cycloalkyl)(C₁₋₈ alkyl), OH, NH₂, SH, SO₂(C₁₋₈ alkyl), P(═O)(OC₁₋₈ alkyl)(C₁₋₈ alkyl), P(═O)(OC₁₋₈ alkyl)₂, C≡C—(C₁₋₈ alkyl), C≡CH, CH═CH(C₁₋₈ alkyl), C(C₁₋₈ alkyl)═CH(C₁₋₈ alkyl), C(C₁₋₈ alkyl)═C(C₁₋₈ alkyl)₂, Si(OH)₃, Si(C₁₋₈ alkyl)₃, Si(OH)(C₁₋₈ alkyl)₂, C(═O)(C₁₋₈ alkyl), CO₂H, halogen, CN, CF₃, CHF₂, CH₂F, NO₂, SF₅, SO₂NH(C₁₋₈ alkyl), SO₂N(C₁₋₈ alkyl)₂, S(═O)NH(C₁₋₈ alkyl), S(═O)N(C₁₋₈alkyl)₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl), NHSO₂N(C₁₋₈ alkyl)₂, and NHSO₂NH₂; and q is an integer greater than or equal to 1.

In further aspects, the L is a polyethyleneglycol optionally substituted with aryl or phenyl, having from 1 to 10 ethylene glycol units. In some aspects, the L is a polyethylene glycol having from 3 to 5 ethylene glycol units. In some aspects, the L is a polyethylene glycol having 4 ethylene glycol units.

In further aspects, the L is a multi-arm, or branched, polyethyleneglycol optionally substituted with aryl or phenyl, and therefore the compound has one or more E3LBM covalently bound to one or more arms of the L, and the compound has one or more FOXP3BP covalently bound to another one or more arms of the L. In one aspect, the L is a four-arm polyethyleneglycol, optionally substituted with aryl or phenyl and having from 1 to 10 ethylene glycol units in each arm; and the compound has the E3LBM covalently bound to each one of three arms of the L, and the FOXP3BP covalently bound to the fourth arms of the L. In another aspect, the L is a four-arm polyethyleneglycol, optionally substituted with aryl or phenyl and having from 1 to 10 ethylene glycol units in each arm; and the compound has the E3LBM covalently bound to each one of two arms of the L, and the FOXP3BP covalently bound to each of the other two arms of the L. In yet another aspect, the L is a four-arm polyethyleneglycol, optionally substituted with aryl or phenyl and having from 1 to 10 ethylene glycol units in each arm; and the compound has the E3LBM covalently bound to one of the arms of the L, and the FOXP3BP covalently bound to each of the other three arms of the L. In yet another aspect, the L is a 3, 4, 5, 6, 7, 8, 9, 10, 12, 16, or m-arm polyethyleneglycol, said m is an integer greater than or equal to 2, optionally substituted with aryl or phenyl, and the compound has an E3LBM covalently bound to each of 1, 2, 3, 4, or x number of arms of the L, as well as an FOXP3BP covalently bound to each of 1, 2, 3, 4, or y number of arms of the L, wherein x and y are independent integers greater than or equal to 1, and wherein x+y≤m.

Pharmaceutically acceptable salts, enantiomers, stereoisomers, solvates, polymorphs, or prodrugs of a compound of a chemical structure of E3LBM-L-FOXP3BP are also provided.

Further embodiments provide that the compound is provided with an mTOR inhibitor, a PD-1 inhibitor, a PD-L1 inhibitor, or another immune checkpoint inhibitor, a CD4 lymphocyte depleting agent, immunomodulating drugs (or IMiDs), or a combination thereof. Examples of drugs that target PD-1 include Pembrolizumab (Keytruda), Nivolumab (Opdivo), Dostarlimab, and Cemiplimab (Libtayo). Examples of drugs that target PD-L1 include Atezolizumab (Tecentriq), Avelumab (Bavencio), and Durvalumab (Imfinzi). Examples of drugs that target checkpoint protein CTLA-4 include Ipilimumab (Yervoy) and tremelimumab (Imjuno). An example of a drug that targets checkpoint protein LAG-3 includes Relatlimab. Examples of IMiDs include Thalidomide (Thalomid), lenalidomide (Revlimid), and pomalidomide (Pomalyst).

Some embodiments provide that the compound is provided with an mTOR inhibitor for use in treating a subject with cancer. Some embodiments provide that the compound is provided with an mTOR inhibitor and a PD-1 inhibitor for use in treating a subject with cancer. Other embodiments provide that the compound is not provided with an immune check point inhibitor, as the compound itself or with another agent such as an mTOR inhibitor is effective for treating a subject with a cancer. Further embodiments provide that the compound is not provided with P60 or a variant thereof that can bind to FOXP3 in a use to treat a subject with cancer.

As described herein, mTOR inhibitors may be any one or more of a small molecule, a peptide, an antibody or a fragment thereof, a nucleic acid molecule and/or a macrolide compound. In an embodiment, the antibody specifically binds mTOR so as to inhibit mTOR. The antibody may be any one or more of a monoclonal antibody or fragment thereof, a polyclonal antibody or a fragment thereof, a chimeric antibody, a humanized antibody, a human antibody or a fragment thereof, or a single chain antibody. These antibodies can be from any source, e.g., rat, mouse, guinea pig, dog, cat, rabbit, pig, cow, horse, goat, donkey or human. Fragments of antibodies may be any one or more of Fab, F(ab′)2, Fv fragments or their fusion proteins. An exemplary mTOR inhibitor is a macrolide compound. Examples of macrolide compounds include but are not limited to temsirolimus (CCI-779), evirolimus (RAD-001), and/or sirolimus (rapamycin), or a pharmaceutical equivalent, analog, derivative or a salt thereof.

A PD-1 inhibitor may be any one or more of an antibody, a fragment thereof, a small molecule, a peptide, or a nucleic acid molecule. For example, an anti-PD-1 antibody may be any one or more of a monoclonal antibody or fragment thereof, a polyclonal antibody or a fragment thereof, a chimeric antibody, a humanized antibody, a human antibody or a fragment thereof, or a single chain antibody. These antibodies can be from any source, e.g., rat, mouse, guinea pig, dog, cat, rabbit, pig, cow, horse, goat, donkey or human. Fragments of antibodies may be any one or more of Fab, F(ab′)2, Fv fragments or their fusion proteins.

As described herein, immune checkpoint inhibitors may be an antibody against PD-1, an antibody against PD-L1, an antibody against PD-L2, an antibody against CTLA-4, an antibody against KIR, an antibody against IDO1, an antibody against IDO2, an antibody against TIM-3, an antibody against LAG-3, an antibody against OX40R, and an antibody against PS, or a combination thereof.

Methods for inhibiting a T-cell are also provided, which include a step of contacting a T-cell with a compound having a chemical structure of E3LBM-L-FOXP3BP, wherein the T-cell expresses FOXP3. In various aspects, the T-cell is a regulatory T-cell, and the contact inhibits the regulatory T-cell. In various aspects, the T-cell is CD4+. In further aspects, said inhibited T-cell is in (proximity to) a population of cells that also contain engineered T-cells which express or comprise a nucleotide sequence coding for a chimeric antigen receptor (CAR) directed against at least an antigen expressed at the surface of a malignant or infected cell.

Methods are also provided for inhibiting growth of a tumor in a subject, or treating the subject having a cancer, which include the step of administering to the subject a therapeutically effective amount of a compound having a chemical structure of E3LBM-L-FOXP3BP.

In various implementations, the methods for inhibiting tumor growth or treating a subject suffering from a cancer further include administering to the subject a therapeutically effective amount of an mTOR inhibitor, a PD-1 inhibitor, or another immune checkpoint inhibitor, a CD4 lymphocyte depleting agent, or a combination thereof. In further embodiments, the methods for inhibiting tumor growth or treating a subject suffering from a cancer include or consist of administering to the subject a therapeutically effective amount of a compound having a chemical structure of E3LBM-L-FOXP3BP and an effective amount of an mTOR inhibitor. In various implementations, the methods do not include administering FOXP3BP (a peptide capable of binding to FOXP3) to the subject.

Additional methods are provided for improving an anti-tumor effect of an immuno-oncology drug in a subject, which includes administering to the subject a therapeutically effective amount of a compound having a chemical structure of E3LBM-L-FOXP3BP. An immuno-oncology drug includes but is not limited to an mTOR inhibitor, a PD-1 inhibitor, or another immune checkpoint inhibitor, a CD4 lymphocyte depleting agent, or a combination thereof.

Examples of cancer include but are not limited to, carcinoma, blastoma, and sarcoma. More particular examples of such cancers include, but are not limited to, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas.

In various aspects, the treatment methods result in a smaller tumor in the treated subject, or a smaller growth of the tumor (e.g., the tumor size growth is a smaller percentage relative to the initial tumor size) in the treated subject, compared to a non-treated subject having the cancer, or compared to a subject having the cancer but treated without the compound having the chemical structure of E3LBM-L-FOXP3BP.

In some aspects, the compound having the structure of E3LBM-L-FOXP3BP is administered to a subject at 100-200 mg/period, 200-300 mg/period, 300-400 mg/period, 400-500 mg/period, 500-600 mg/period, 600-700 mg/period, 700-800 mg/period, 800-900 mg/period, 900-1000 mg/period, 1000-1100 mg/period, 1100-1200 mg/period, 1200-1300 mg/period, 1300-1400 mg/period, 1400-1500 mg/period, 1500-1600 mg/period, 1600-1700 mg/period, 1700-1800 mg/period, 1800-1900 mg/period or 1900-2000 mg/period. A dosing period can be daily (per day), weekly, monthly, quarterly, annually, or as determined by a medical professional. In various embodiments, the compound is administered at 0.1-0.5 mg/kg, 0.5-1.0 mg/kg, 1.0-1.5 mg/kg, 1.5-2.0 mg/kg, 2.0-2.5 mg/kg, 2.5-5 mg/kg, 5-10 mg/kg, 10-15 mg/kg, 15-20 mg/kg, 20-25 mg/kg, 25-30 mg/kg, 30-35 mg/kg, 35-40 mg/kg, 40-45 mg/kg, 45-50 mg/kg, 50-55 mg/kg, 55-60 mg/kg, 60-65 mg/kg, 65-70 mg/kg, 70-75 mg/kg, 75-80 mg/kg, 80-85 mg/kg, 85-90 mg/kg, 90-95 mg/kg or 95-100 mg/kg.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

We created and tested a PROTAC (proteolysis targeting chimera) drug that inhibits FoxP3 expressing regulatory T cells. This drug releases the “brakes” on the immune system. The resulting immune activation can be used as a therapy for cancer or infection.

A PROTAC includes two active domains connected by a linker. One domain binds the target protein. The other domain engages an E3 ubiquitin ligase, causing the target protein to be ubiquitinated and tagged for proteasomal degradation.

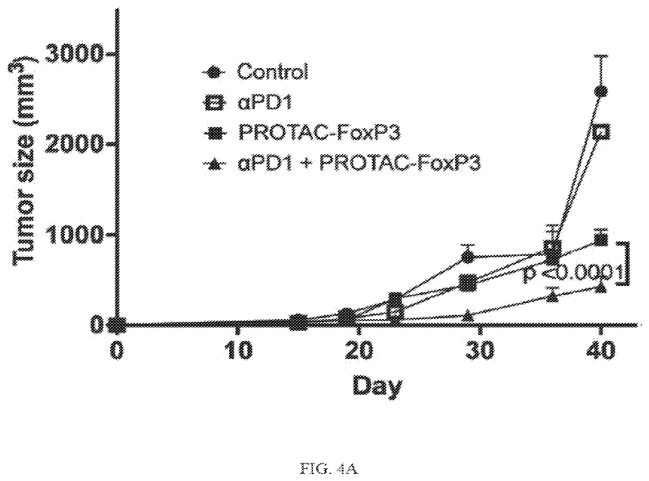

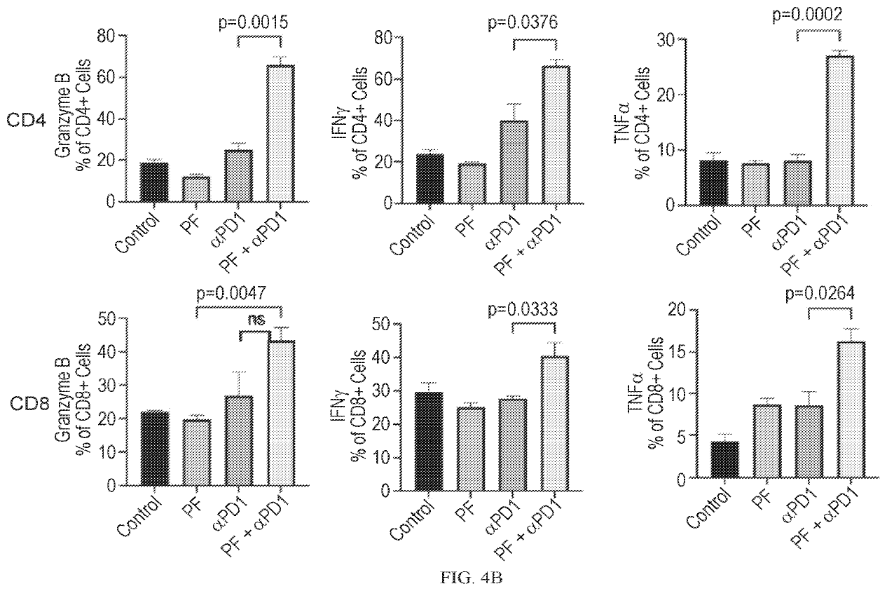

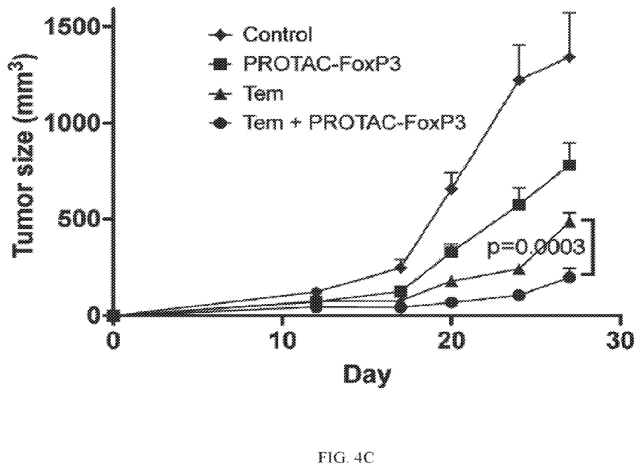

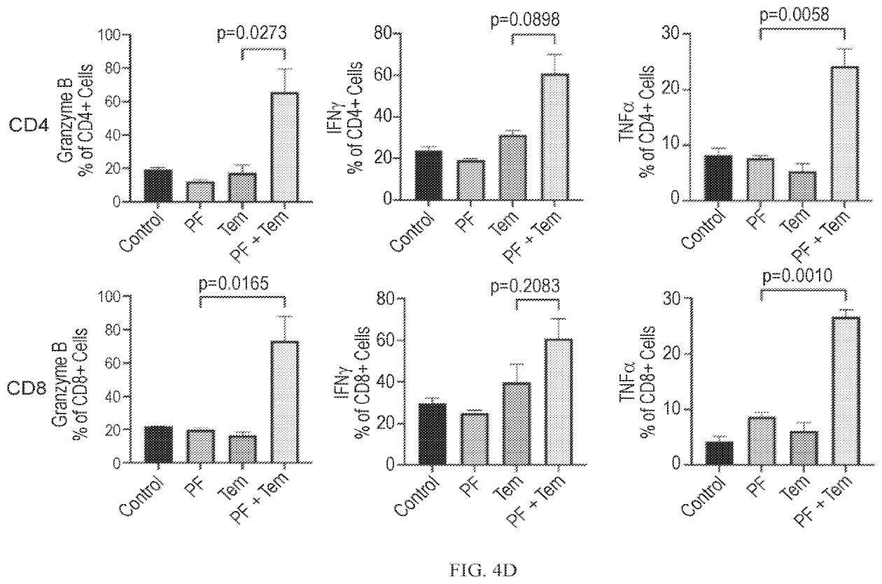

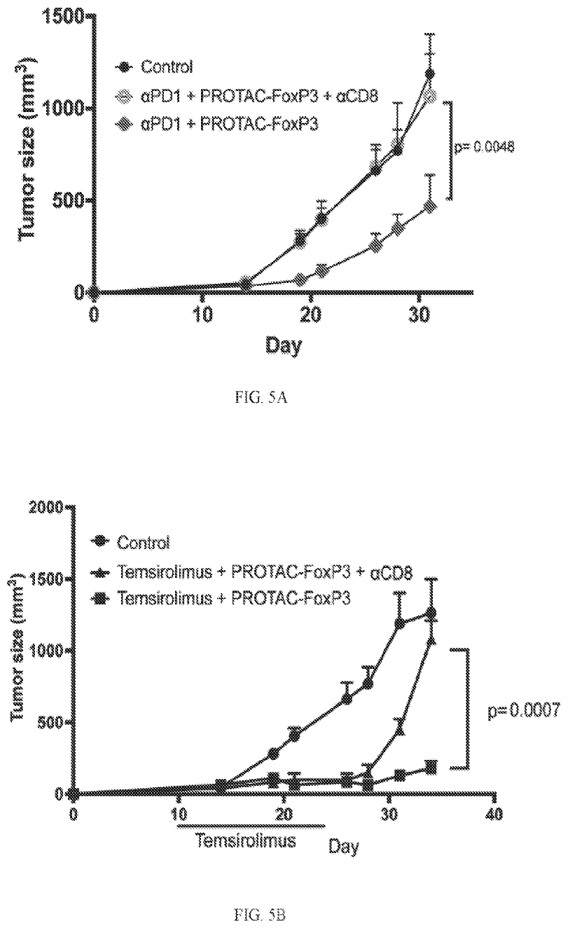

Peptide P60 (RDFQSFRKMWPFFAM, SEQ ID NO:1) is a 15-mer synthetic peptide that binds FoxP3. P60 was synthesized and linked to a ligand of an E3 ubiquitin ligase, pomalidomide, to create a PROTAC drug denoted as P-FoxP3 (Pomalidomide-PEG₄-RDFQSFRKMWPFFAM). P-FoxP3 was synthesized starting with pomalidomide-PEG₄-COOH (CAS #2138440-81-8; or synthesized as shown in FIG. 4A) and P60 peptide (often prepared via solid phase peptide synthesis), via conjugation reaction/procedure (shown in FIG. 4B).

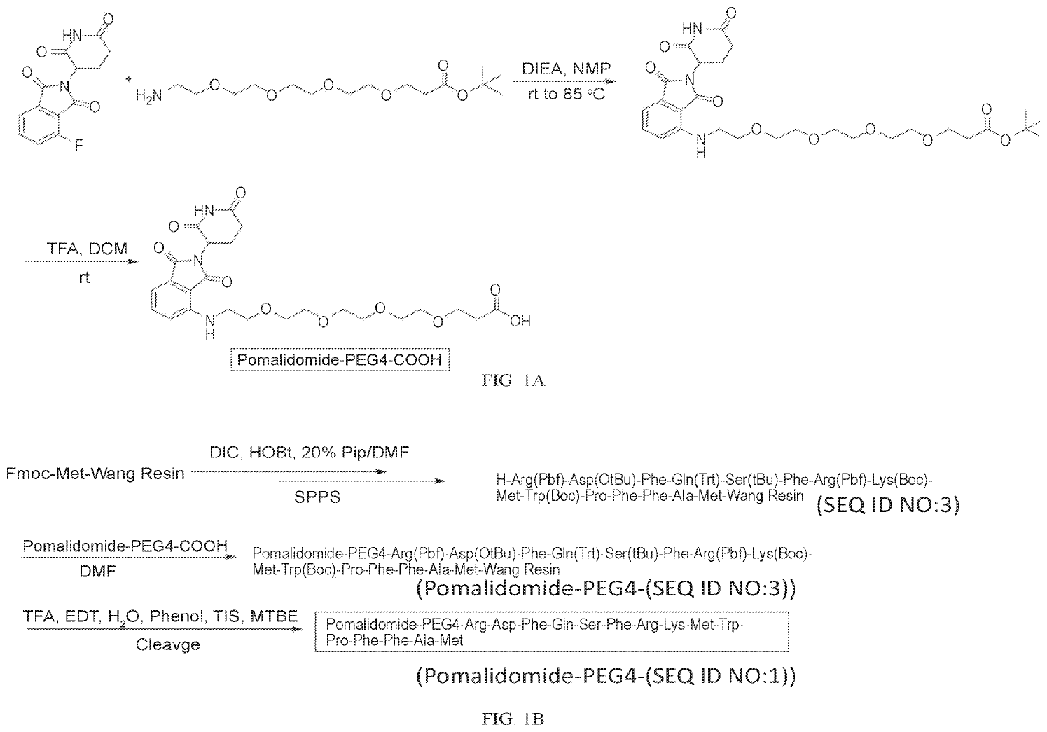

P-FoxP3 was designed to bind FoxP3 and cereblon (E3 ubiquitin ligase). A surface plasmon resonance (SPR) assay confirmed that P-FoxP3 forms a ternary complex with cereblon and FoxP3 (FIG. 1A).
Equilibrium dissociation constant, KD ternary: 4.34×10⁻⁷ M
KD binary: 9.87×10⁻⁷ M
Cooperativity, a=KD binary/KD ternary=9.87×10⁻⁷ M/4.34×10⁻⁷ M=2.27
   wherein a>1, positively cooperative;
      a=1, non-cooperative;
      a<1, negatively cooperative.

### Example 1

### Materials and Methods

### Synthesis of P60 Peptide and PROTAC for FoxP3 (PF)

Pomalidomide was conjugated to the N-terminal of the P60 peptide (RDFQSFRKMWPFFAM, SEQ ID NO:1) through a bifunctional polyethylene glycol (PEG)₄ linker (PF). Pomalidomide-(PEG)₄ conjugate (PC) was synthesized by BOC Sciences (Shirley, NY). PF and PC were purified to ≥98% purity, and the structure was confirmed by electrospray ionization (ESI) mass spectroscopy prior to purchase. P60 peptide was synthesized in the Cedars Sinai institutional core. The peptide (RDFQSFRKMWPFFAM, SEQ ID NO:1) was synthesized by the solid-phase method via Fmoc strategy, using 2-(6-Chloro-1-H-benzotriazole-1-yl)-1,1,3,3-tetramethylaminium hexafluorophosphate (HCTU) and diisopropylethylamine (DIEA) as the coupling agents on a CS136XT automated peptide synthesizer.

The SPR assay for ternary complex cooperativity was performed by BOC Sciences who provided the following details: Various concentrations of cereblon were dissolved in water and manually printed onto the 47 nm gold-coated PlexArray Nanocapture Sensor Chip (Plexera Bioscience, Seattle, WA) at 40% humidity. Each concentration was printed in replicate, and each spot contained 0.2 L of sample solution. The chip was incubated overnight in 80% humidity at 4° C., and rinsed with 10×PBST for 10 minutes, 1×PBST for 10 minutes, and deionized water twice for 10 minutes. The chip was then blocked with 5% non-fat milk in water overnight, washed as before, and then dried under a stream of nitrogen.

SPR imaging measurements were performed with PlexAray HT (Plexera Bioscience, Seattle, WA, US). Buffers and samples were injected by a non-pulsatile piston pump into the 30 μL flow cell. Each measurement cycle contained four steps: washing with PBST running buffer at a constant rate of 2 L/s to obtain a stable baseline, sample injection at 5 μL/s for binding, surface washing with PBST at 2 L/s for 300 s, and regeneration with 0.5% H₃PO₄ at 2 L/s for 300 s. All the measurements were performed at 25° C. The signal changes after binding and washing (in AU) are recorded as the assay value. Selected protein-grafted regions in the SPR images were analyzed, and the average reflectivity variations of the chosen areas were plotted as a function of time. Real-time binding signals were recorded and analyzed by Data Analysis Module (DAM, Plexera Bioscience, Seattle, WA, US). Kinetic analysis was performed using BIAevaluation 4.1 software (Biacore, Inc.).

### Antibody and Reagents

The following monoclonal antibodies (mAb) with a fluorescent conjugate for flow cytometry were obtained from Biolegend (San Diego, CA): αCD4 (GK 1.5 and RM4-5), αCD8 (53-6.7), αT-bet (4B10), αCD62L (MEL-14), αFoxP3 (FJK-16s), αIFN-γ (XMG1.2), αTNFα (TN3-19.12). αCD8 (2.43) for T cell depletion was purchased from BioXcell (West Lebanon, NH). Brefeldin A was purchased from eBioscience/ThermoFisher Scientific (San Diego, CA). PMA was purchased from Selleck Chemicals LLC (Houston, TX).

### Tumor Cell and Mice

RENCA mouse kidney cancer cell line was purchased from ATCC (Manassas, Virginia) and maintained in RPMI 1640 medium supplemented with 10% heat-inactivated FBS (Gemcell, West Sacramento, CA), 2 mmol/L of L-glutamine, 100 units/mL of penicillin, 100 μg/mL of streptomycin, and 250 ng/mL of Amphotericin B (ThermoFisher, Waltham, MA). These cells were periodically authenticated by morphologic and histologic inspection, and animal grafting to assess their ability to grow and metastasize. The cells were annually tested for mycoplasma using MycoAlert Kit (Lonza, Allendale NJ).

Mice: Balb/C mice, 5-8 weeks old, were purchased from Jackson laboratory (Ellsworth, Maine) and housed under pathogen free conditions. All experiments involving animals followed federal and state standards, which include the federal Animal Welfare Act and the NIH guide for the care and use of laboratory animals. The RENCA tumors were generated by subcutaneously injecting 2×10⁵ tumor cells into the frank. The day tumor cells were injected was considered day 0. PF (0.1 mmol), PC (0.1 mmol) or P60 (100 ug) was administered i.p. twice a week starting on day 10; αPD1 (200 μg) was administered i.p. twice a week starting on day 5; temsirolimus (15 μg) was administered i.p. every other day, starting on day 10. All drugs were administered throughout the experimental course unless indicated. CD8 cells were depleted with a one-time αCD8 (200 μg) i.p. administration on day 2.

### Dendritic Cell Vaccine and T Cells Stimulation

To prepare a dendritic cell (DC) vaccine, mouse bone marrow was harvested from C57BL/6 mice and suspended in RPMI supplemented with 10% FBS. GM-CSF (10 ng/mL) was added to the medium and placed at 37° C. with 5% CO₂. RPMI medium with GM-CSF was replaced every other day. DCs were harvested on day 7 and pulsed with tumor cell lysate for 16 hours and activated with 10 μg/mL CpG for 4 hours. For in vitro activation of CD8 cells, DCs pulsed with tumor lysate were washed with PBS and cocultured with enriched CD8 cells (1:5 ratio of DC:CD8) for 3 days before being used for adoptive transfer to mice.

### T Cell Characterization.

To assess T cell activation in vitro, single suspension of mouse splenocytes were separated into CD4 positive (+) and negative (−) cells using EasySep Mouse CD4+ selection kit (STEMCELL technologies, Vancouver, BC, Canada). CD4+ cells contain Tregs and were treated with varying concentrations of PC, P60 or PF for 72 hours (treated-Treg). To study CD8+ cell proliferation, CD4− cells, which contain CD8+ effector cells, were carboxyfluorescein succinimidyl ester (CFSE) labeled to assess CFSE dilution in the presence of treated-Treg. To study effector cell function, CD4+ and CD4− cells were cocultured (1.2 ratio) with treated-Treg and stimulated with αCD3 (10 μg/mL) and αCD28 (5 μg/mL) for 3 days; percent of CD4+ or CD8+ cells staining for intracellular FoxP3, CFSE, or TNFα were assessed by flow cytometry (LSR II, BD Biosciences, San Jose, CA 95131).

To assess T cells in mice, CD4+ FoxP3+ cells from serial peripheral blood draws were assessed by flow cytometry. To assess lymphocytes in mice treated with various therapies, lymph nodes and spleens were harvested to make single-cell suspensions that were activated by a CA9 peptide (AYEQLLSHL) for 4 days, and then flow cytometry was used to assess CD4, CD8, IFNγ, TNFα, CD62L CD44, and Eomes.

### Human Lymphocytes

Human peripheral blood mononuclear cells (PBMC) were treated with phorbol 12-myristate 13-acetate (PMA) (10 ng/ml) and Ionomycin (2 μg/ml) to increase FoxP3 expression. Human CD4+ PBMC were isolated using EasySep Human CD4+ selection kit (STEMCELL technologies). When treating cells with PF or various controls (all 10 μM), lymphocytes were activated with αCD3 (10 μg/mL) and αCD28 (5 μg/mL) for 3 days. CD8 proliferation was measured using a CFSE dilution assay. Immune activation was assessed by measuring TNFα or IFNγ in CD8+ or CD4+ T cells using flow cytometry.

### Results

### A Proteolysis-Targeting Chimera (PROTAC) Targeting FoxP3 (PF).

To create a FoxP3 targeting PROTAC, pomalidomide, a derivative of thalidomide and used for treating multiple myeloma, was conjugated to the N-terminal of a polypeptide that binds FoxP3 (P60) through a bifunctional polyethylene glycol (PEG)₄ linker (PF; FIGS. 1A and 1B). Peptide P60 (RDFQSFRKMWPFFAM, SEQ ID NO:1) is a 15-mer synthetic peptide that binds FoxP3. P60 was synthesized and linked to a ligand of an E3 ubiquitin ligase, pomalidomide, to create a PROTAC drug denoted as P-FoxP3 (Pomalidomide-PEG4-RDFQSFRKMWPFFAM (SEQ ID NO:1)). P-FoxP3 was synthesized starting with pomalidomide-PEG4-COOH (CAS #2138440-81-8; or synthesized as shown in FIG. 1A) and P60 peptide (often prepared via solid phase peptide synthesis), via conjugation reaction/procedure (shown in FIG. 1).

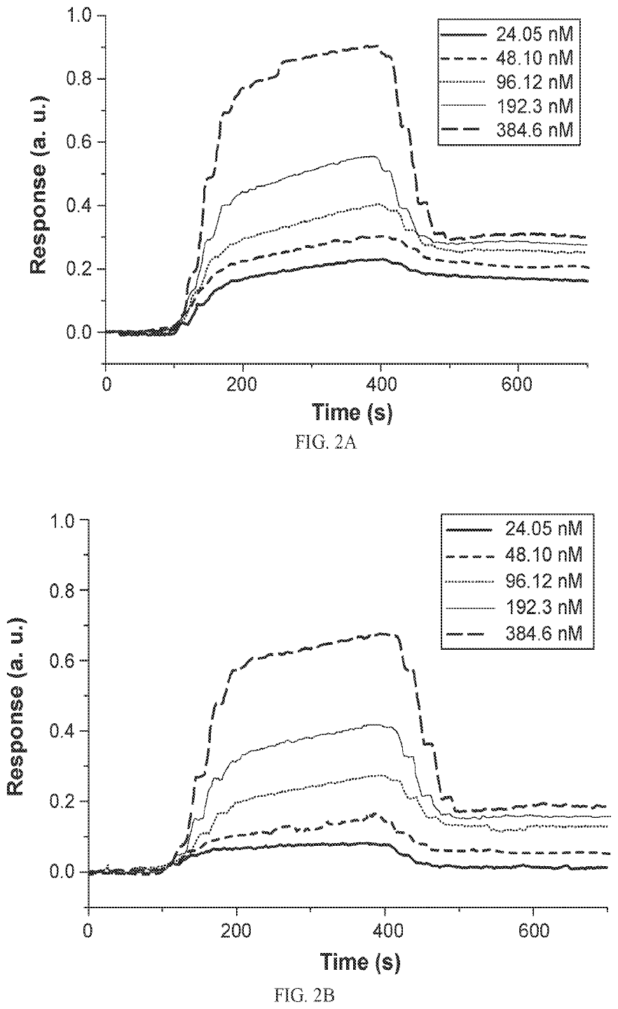

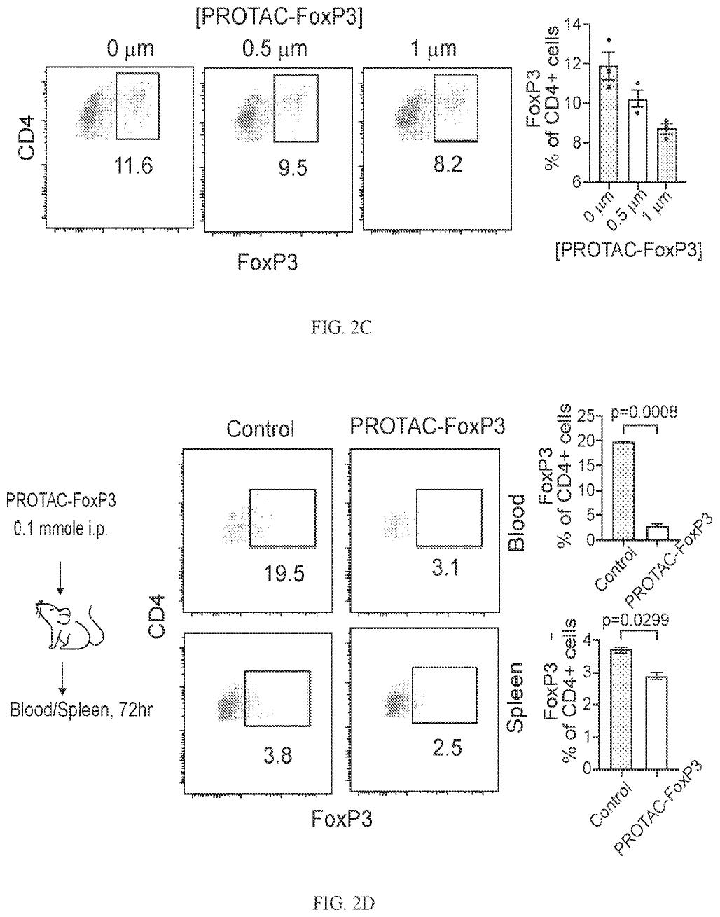

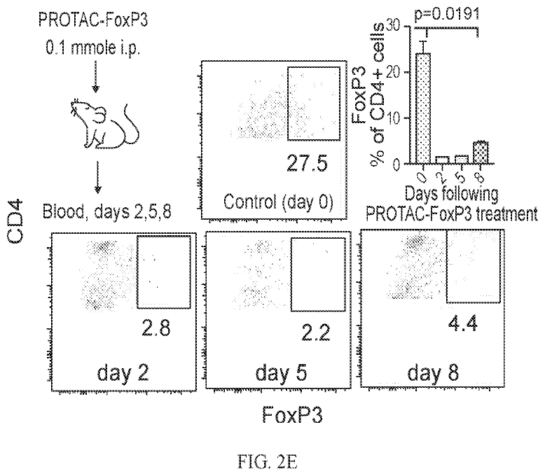

The structure of PF was confirmed by electrospray ionization mass spectroscopy. Surface plasmon resonance (SPR) assay was used to measure the equilibrium dissociation constant (KD) for the binary complex consisting of cereblon and PF (FIG. 2B) and the KD for the ternary complex consisting of cereblon, FoxP3, and P-FoxP3 (FIG. 2A). The SPR assay confirmed that PF forms ternary complexes with recombinant FoxP3 and cereblon, which is an E3 ligase that pomalidomide binds to activate the ubiquitin-proteasome system against FoxP3 (FIGS. 2A and 2B). Equilibrium dissociation constant, KD ternary: 4.34×10⁻⁷ M (FIG. 2A); and KD binary: 9.87×10⁻⁷ M (FIG. 2B).

Cooperativity, a=KD binary/KD ternary=9.87×10⁻⁷ M/4.34×10⁻⁷ M=2.27, wherein a>1, positively cooperative; a=1, non-cooperative; and a<1, negatively cooperative. The cooperativity factor (K_{D} binary/K_{D} ternary) was 2.27, cooperativity factor >1 indicating positive cooperativity and formation of ternary complexes.

To determine if PF results in the degradation of FoxP3, mouse splenocytes were treated in vitro with varying concentrations of PF and percent of CD4+ cells staining for intracellular FoxP3 was assessed by flow cytometry. As expected, FoxP3 levels in CD4+ cells decreased in a dose-dependent manner (FIG. 2C). The next step was to determine if PF can decrease FoxP3 in vivo. A single dose of PF was administered to mice. 72 hours later, FoxP3 positive CD4+ cells in peripheral blood and spleen were assessed by flow cytometry. FoxP3 in CD4+ cells were decreased 3 days later in both spleen and peripheral blood, and the peripheral decrease was highly significant (p<0.0008; FIG. 2D). To understand the time course of FoxP3 decrease, serial blood draws were assessed after a single injection of PF (FIG. 2E). FoxP3 positive CD4+ cells in peripheral blood were assessed by flow cytometry at various timepoints following the PF administration. FoxP3 in CD4+ cells immediately dropped as measured on day 2, and slightly came up on day 8. In summary, a PROTAC targeting FoxP3 (PF) degraded FoxP3 in vitro and in vivo.

### PF Decreased the Immunosuppressive Function of Tregs.

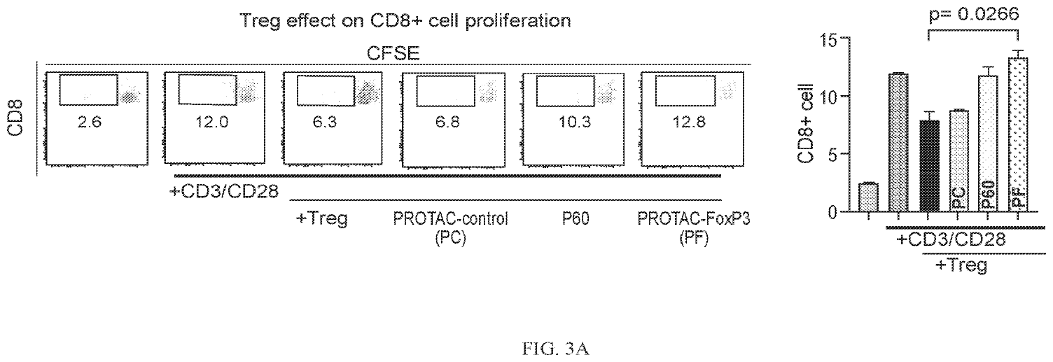

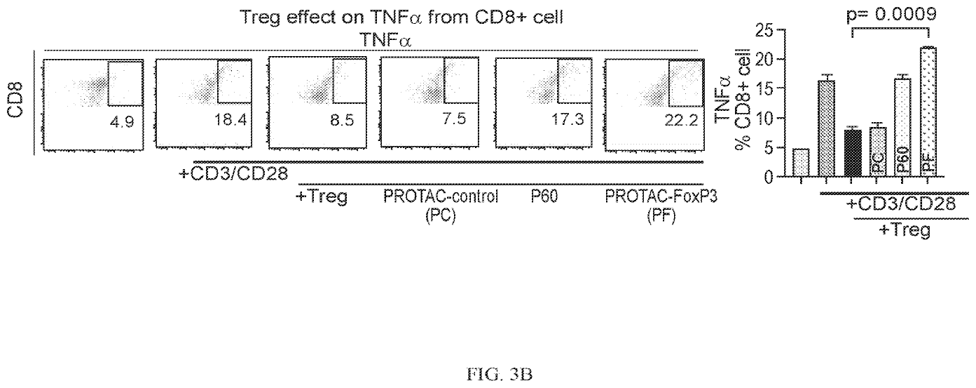

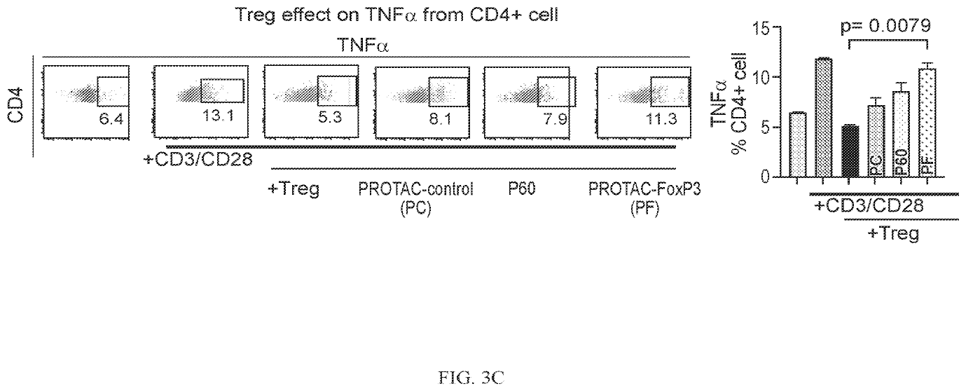

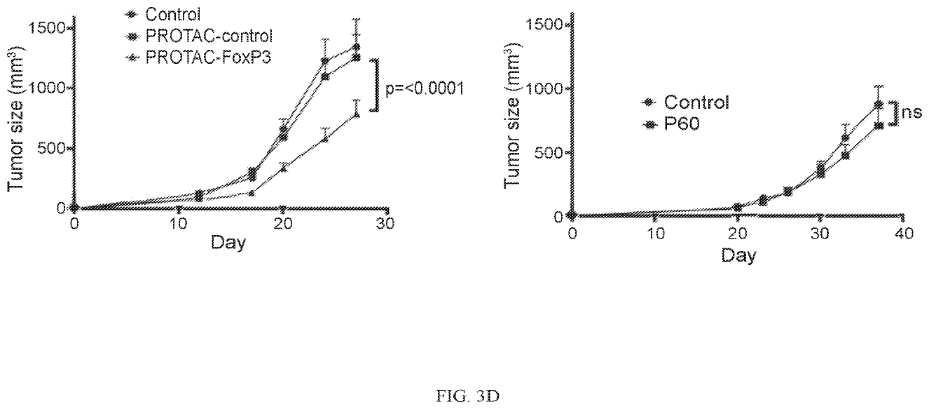

Tregs play a central role in suppressing and regulating the immune response. FoxP3 is a transcription factor and the master regulator of Treg lineage and suppressive function. Therefore, the next step was to determine if PF can suppress Treg function and increase CD8+ lymphocyte activity. To treat Tregs, CD4+ splenocytes were isolated and treated with PF or various controls for 72 hours. To assess the immunosuppressive effects of these cells, they were washed and co-cultured with effector cells, which were stimulated with antibodies to CD3 and CD28 in vitro. CD8 proliferation was measured using a CFSE dilution assay. To assess CD8+ effector cells, CD4 negative splenocytes were CFSE labeled and CFSE dilution was measured in CD8+ cells by flow cytometry (FIG. 3A). CD8+ lymphocyte activation was assessed by measuring TNFα (FIG. 3B). Also, CD4+ lymphocyte activation was assessed by measuring TNFα (FIG. 3C). In co-culture studies, PF treated CD4+ lymphocytes, which include Tregs, were less capable of suppressing CD8+ lymphocyte proliferation (FIG. 3A) or activation of CD8+ lymphocytes as measured by TNFα expression (FIG. 3B). Similarly, PF treated CD4+ cells were less capable of suppressing the activation of CD4+ effector lymphocytes (FIG. 3C). PC (PROTAC control), which are PF without P60, and P60 were used as two separate negative controls.

In cancer, Tregs can suppress the antitumor immune response, which is primarily mediated by CD8+ lymphocytes. Therefore, PF was administered to mice to enhance antitumor immunity and suppress growth of syngeneic mouse kidney cancer, which is considered an immunoresponsive cancer type. Mice tumors were established by injecting RENCA cells subcutaneously in the flank of Balb/C mice (5-7 mice per group). RENCA cells were injected subcutaneously to establish palpable tumors, and 10 days later mice were treated i.p., twice a week throughout the experiment, with PF, PC, P60 or Saline (FIG. 3D). Pomalidomide conjugated to (PEG)4 (PROTAC-control; PC) and P60 served as controls. Only PF produced a decrease in tumor growth. In summary, PF decreased the immunosuppressive function of Tregs in vitro and decreased tumor growth in mice.

PF Enhanced the Antitumor Immunity Provided by αPD1 or mTOR Inhibition.

Immunotherapies, particularly those targeting PD-1, are standard-of-care for many cancer types. mTOR inhibitors (mTORi) are also approved for many cancer types. Although the mechanism-of-action in patients is still debated, in animal models, mTORi can decrease tumor growth by increasing CD8+ lymphocyte memory formation. In anticipation of clinical testing of PF with αPD1 or mTOR inhibition, we assessed immunotherapy combinations. Palpable RENCA tumors were first established in mice. Mice tumors were established by injecting RENCA-CA9 cells subcutaneously in the flank (day 0) of Balb/C mice (6-8 mice per group). PF was administered i.p., twice a week, throughout the experiment, starting on day 10. αPD1 was administered i.p., twice a week, throughout the experiment, starting on days 5. The combination of PF and αPD1 was more effective in decreasing tumor growth when compared to either drug alone (FIG. 4A). Splenocytes were harvested, and restimulated ex vivo with mouse CA9 peptide. The splenocytes were assessed by flow cytometry for Granzyme B, IFNγ or TNFα after gating on CD4 or CD8. Splenocytes from these mice were assessed for CD8+ lymphocyte response to ex vivo restimulation with a tumor-peptide. The combination therapy produced the strongest granzyme B, IFNγ and TNFα responses in both CD4+ and CD8+ effector cells (FIG. 4B). In a similar mouse tumor growth experiment, temsirolimus was administered i.p. on day 10, every other day, for 14 days. When these experiments were repeated with mTOR inhibitor, temsirolimus (Tem), replacing αPD1, the combination therapy was most effective in decreasing tumor growth (FIG. 4C). When splenocytes were harvested, restimulated and analyzed, the combination therapy was most effective in activating CD4+ and CD8+ cells (FIG. 4D). In summary, PF enhanced the antitumor immunity provided by αPD1 or mTOR inhibition in mice.

### PF-Based Immunotherapy is CD8+ Lymphocyte Dependent.

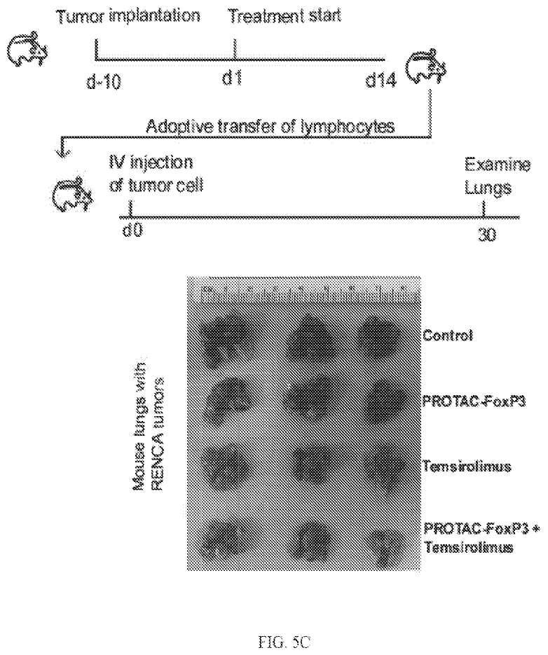

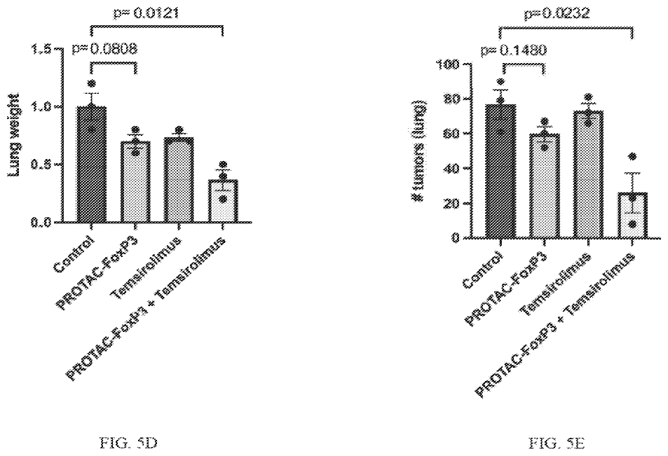

All immunotherapies, particularly when multi-drug combinations are involved, may have off-target effects. For example, drugs may have direct effects on the tumor, inhibiting tumor proliferation or inducing tumor apoptosis. Mice tumors were established by injecting RENCA cell subcutaneously in the flank of Balb/C mice (6-8 mice per group). 10 days later, mice were treated with PF twice per week. CD8 lymphocytes were depleted with αCD8 administered i.p. 2 days after injection of tumor cells. Mice in indicated groups received i.p. αPD1, twice a week, starting day 5 (FIG. 5A). Mice in indicated groups received i.p. temsirolimus every other day for 14 days, starting on days 10 (FIG. 5B). Tumor growth was monitored in these groups.

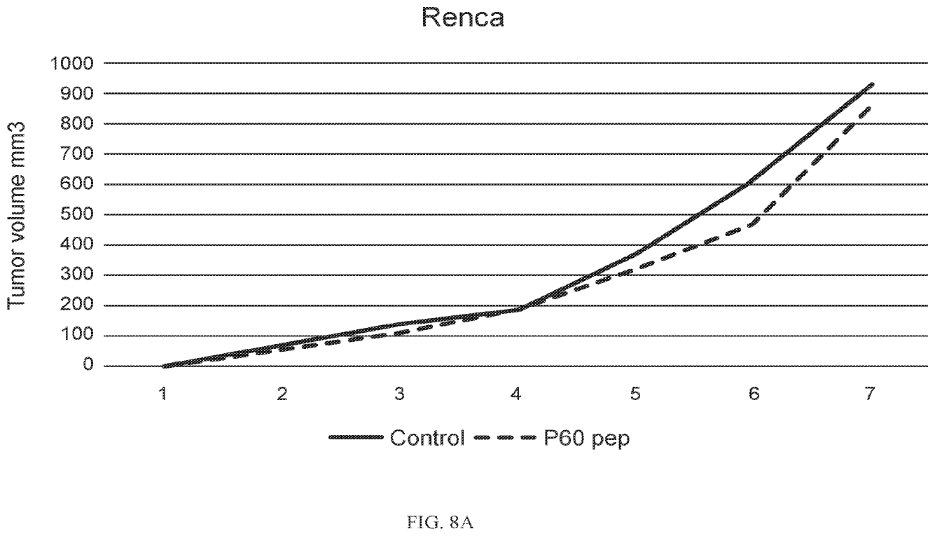

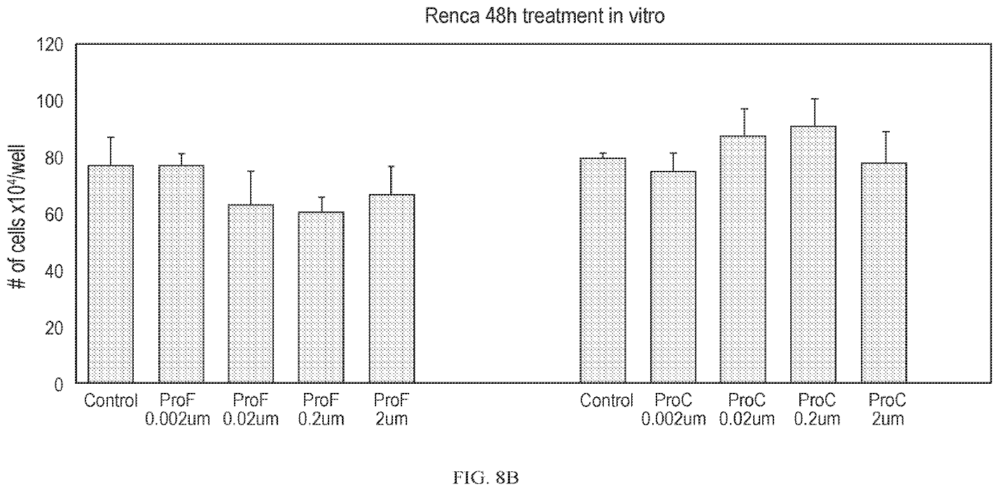

As shown in FIG. 8, P60 alone had no effect on RENCA tumor growth. That is, the tumor in the P60 treated tumor grew at the same rate as the saline treated mice. Further, RENCA cells (20×10⁴/well) were seeded in 12 well plates and treated with PF or PC at 0.002, 0.02, 0.2, or 2 μM. After 48 hours the cells were harvested and counted as shown in FIG. 8B. Although PF did not alter RENCA growth in vitro (FIG. 8B), to firmly establish that our proposed drug combinations have an immune mechanism, we used two separated approaches. The first approach involved depleting CD8+ lymphocytes, which are known to mediate antitumor immunity. CD8+ lymphocyte depletion removed the antitumor mechanism associated with the combination of PF and αPD1 (FIG. 5A). However, CD8+ lymphocyte depletion only partly decreased the antitumor effect of PF, and temsirolimus alone also suppressed tumor growth, suggesting that mTOR inhibition directly decreases tumor growth (FIG. 5B).

Therefore, a second experimental approach was used to characterize immune activation. To determine if immunity is lymphocyte-dependent, RENCA-bearing mice were treated with 4 doses of PF and 7 doses of temsirolimus distributed over 14 days. Spleen and lymph nodes were harvested from these mice, and CD8+ cells were isolated and cultured in vitro with tumor lysate-pulsed DC and IL-2 for 3 days. 2×10⁶ CD8+ cells were adoptively transferred by tail vein injection into recipient mice, which were challenged with i.v. RENCA cells. Lung tumors were examined 30 days later. Lymphocytes from mice treated with PF and temsirolimus were adoptively transferred to treatment-naïve mice, which were challenged with IV RENCA cells (FIG. 5C). The mice receiving the adoptive transfer have never been exposed to PF and temsirolimus so any antitumor effect can be attributed to the transferred lymphocytes. Its notable that this is a very aggressive cancer model since tumor cells injected IV grow rapidly in the lungs and only a very effect therapy can decrease tumor growth. Lung weights and number of tumors visible on the lung surface are shown in FIGS. 5D and 5E, respectively. Histograms are labeled with the treatment provided to the donor mice. The combination therapy significantly decreased tumor growth in the lungs, which is strong evidence of lymphocyte-mediated antitumor immunity (FIGS. 5D and 5E). In summary, in combination therapy with PF plus αPD1 or mTOR inhibition, antitumor immunity was CD8+ lymphocyte dependent.

### PF Enhances Immune Activation and CD8+ Memory Lymphocyte Formation.

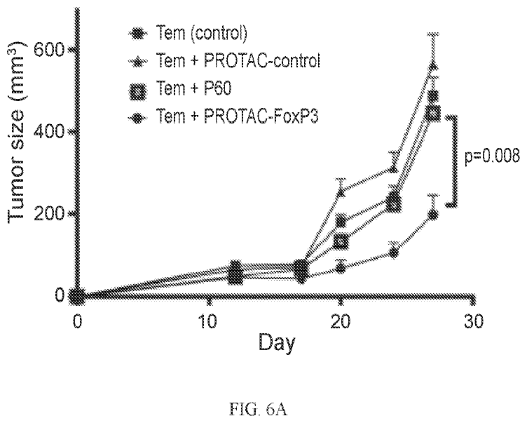

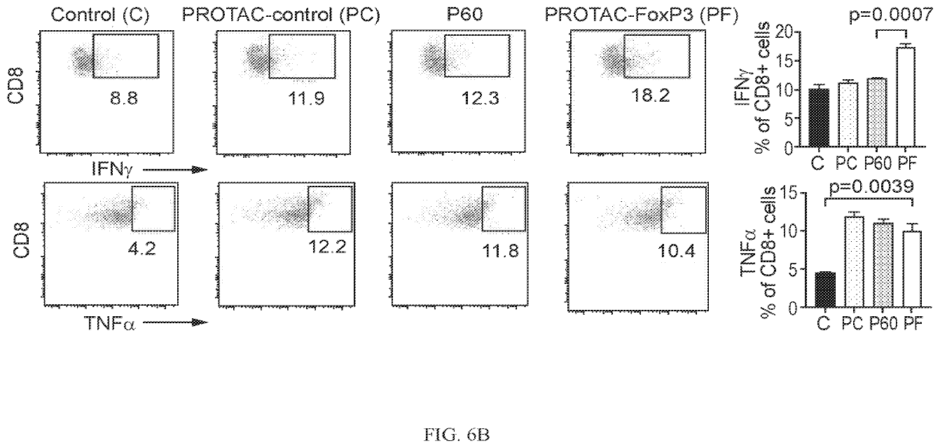

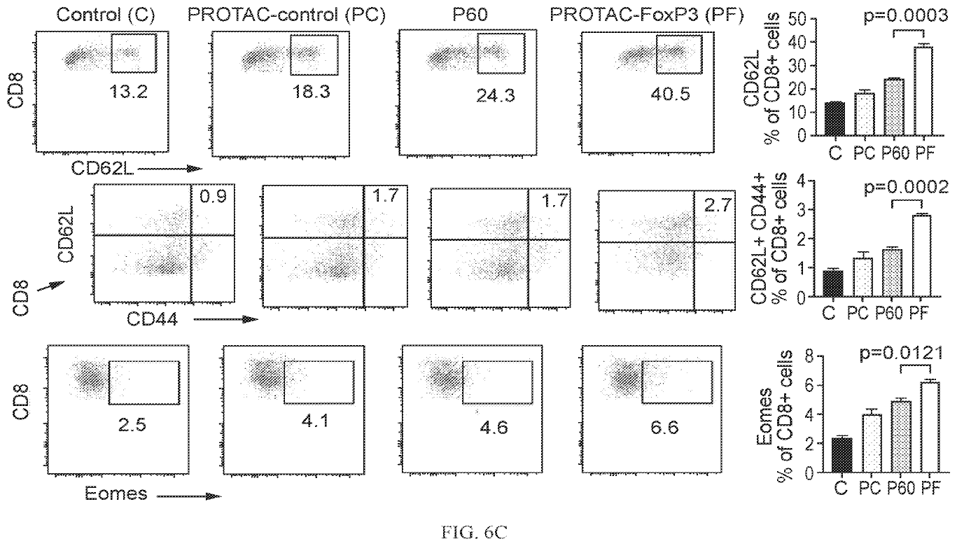

The CD8+ lymphocyte-response to PF and temsirolimus was further characterized. Mice tumors were established by injecting RENCA-CA9 cell subcutaneously in the flank of Balb/C mice (6-8 mice per group), which were treated with the indicated drugs. PF (2×/week, throughout experiment) and temsirolimus (3×/week for 2 wks) treatment administered i.p., starting 10 days after RENCA injection. In a mouse tumor treatment study, the combination was tested again, along with pomalidomide conjugated to (PEG)₄ (PC) and P60 as negative controls (FIG. 6A). The combination of PF and temsirolimus (Tem) was the most effective in suppressing tumor growth. From this experiment, lymphocytes and splenocytes were collected and characterized. Spleen and lymph nodes were harvested on day 28 and restimulated ex vivo with CA9 peptide and assessed by flow cytometry. The combination of PF and temsirolimus (Tem) produced CD8+ cells with greater tumor-specific IFNγ and TNFα responses, i.e., CD8+ lymphocytes treated with PF had increased activation as measured by IFNγ or TNFα, when compared to temsirolimus (Tem) alone (FIG. 6B). Temsirolimus is a mTOR inhibitor, and decreased mTOR signaling triggers CD8+ memory formation. Therefore, we looked at markers of memory, including central memory (CD62L+CD44+) in CD8+ cells (FIG. 6C). PF-treated CD8+ lymphocytes displayed a memory phenotype with increase in CD62L, CD44 and Eomes expression. The combination of PF and temsirolimus (Tem) was most effective in generating memory markers (CD62L, CD44 and Eomes). Therefore, it is clear that PF enhances immune activation and CD8+ memory lymphocyte formation.

### PF Decreases FoxP3 and the Immunosuppressive Function of Human CD4+ Cells.

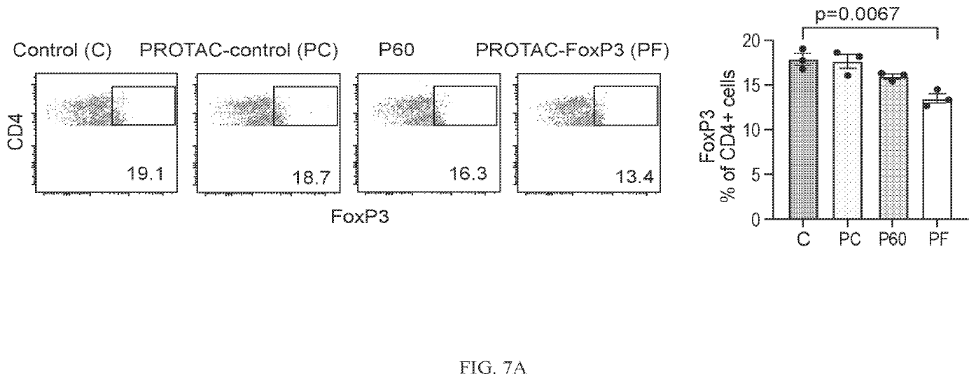

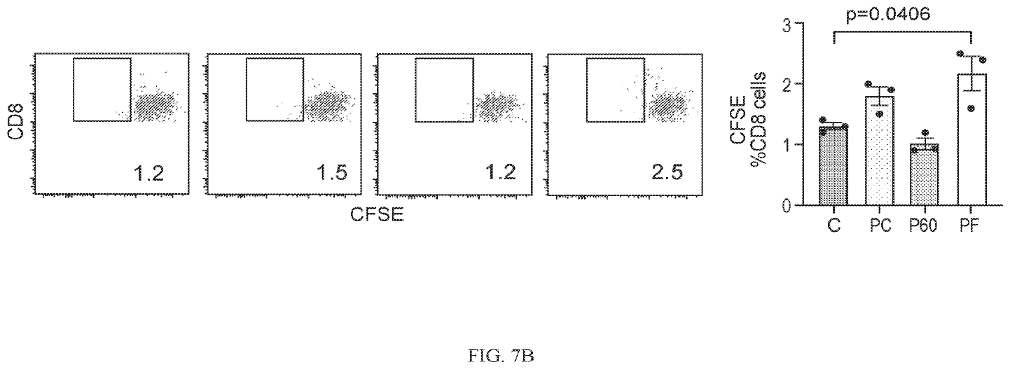

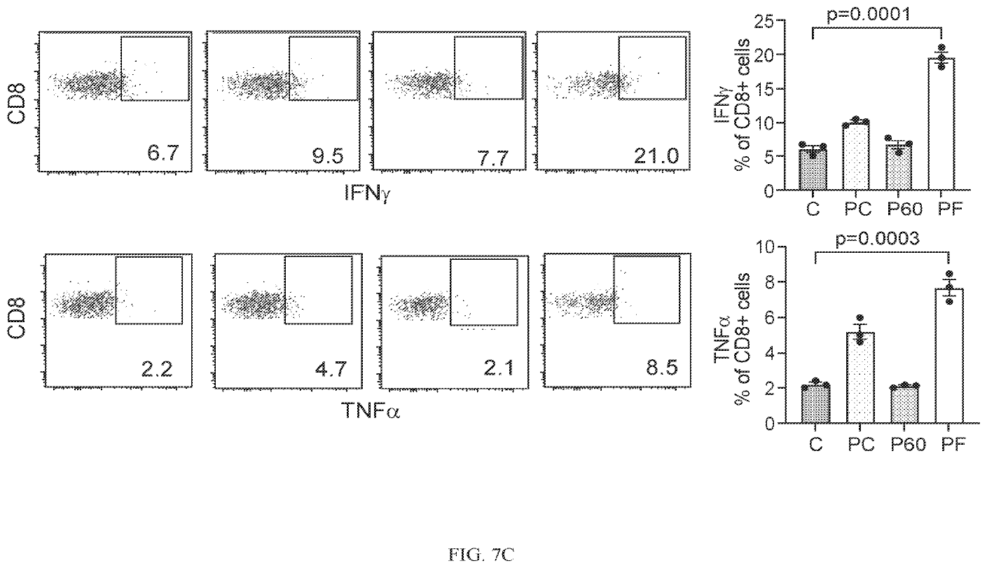

P60 was designed to bind both mouse and human FoxP3. Therefore, we evaluated PF using human peripheral blood mononuclear cells (PBMC). PBMC were treated in vitro with PF (10 μM) or various control drugs (10 μM) for 3 days. PMA 10 ng/ml and Ionomycin 2 μg/ml were added during PF treatment to induce FoxP3. Resulting cells were gated on CD4 and assessed for FoxP3 expression by flow cytometry. PF decreased the percent of CD4+ cells expressing FoxP3 when compared to control, however the difference when compared to P60 was not significant (FIG. 7A). The PBMC were separated into CD4+ and CD4− cells. To treat Tregs, CD4+ PBMC were isolated and treated with PF or various controls for 72 hours. To assess the immunosuppressive effects of these cells, they were washed and co-cultured with CD4− PBMC (effector cells), which were stimulated with antibodies to CD3 and CD28 ex vivo. CD8 proliferation was measured using a CFSE dilution assay (FIG. 7B). To assess CD8+ effector cells, CD4− splenocytes were CFSE labeled and CFSE dilution was measured in CD8+ cells by flow cytometry. CD4+ cells treated with PF were used to suppress the proliferation and activation of CD4− cells, which contained the CD8+ population. In the same experiment, CD8+ cells were assessed for activation by gating on CD8 and assessing IFNγ or TNFα by flow cytometry (FIG. 7C). PF treatment resulted in highest CD8+ lymphocyte proliferation, and greatest enhancement of CD8+ lymphocyte activation as measured by IFNγ or TNFα (FIG. 7C). In all these assays, the differences between PF and P60 were significant. Taken together, these results suggest that PF effectively targets FoxP3 in both mouse and human Tregs.

### Example 2

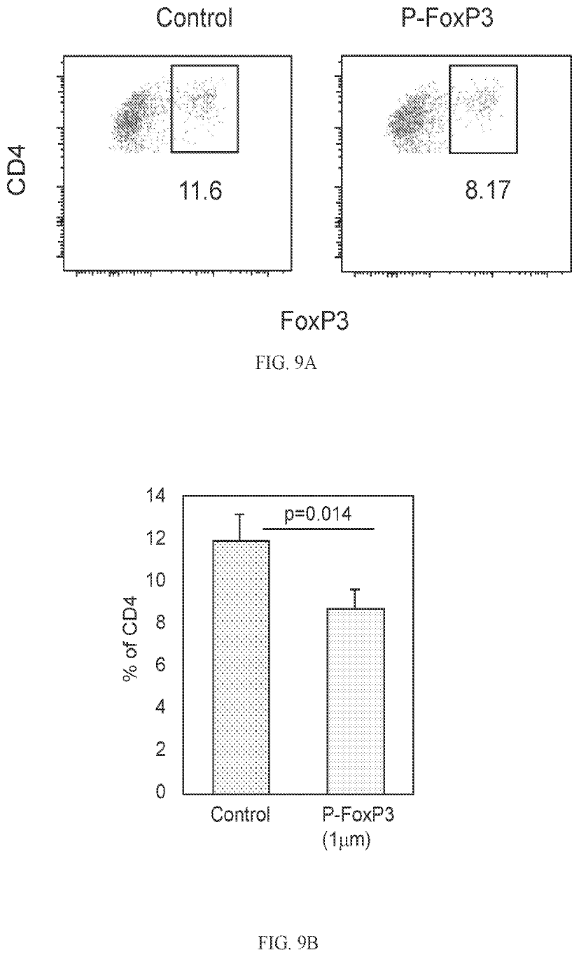

P-FoxP3 degraded FoxP3 in CD4+ cells (FIGS. 9A, 9B). P-FoxP3 was dissolved in ethanol. Fresh mouse splenocytes were cultured in vitro and treated with 1 μm P-FoxP3.

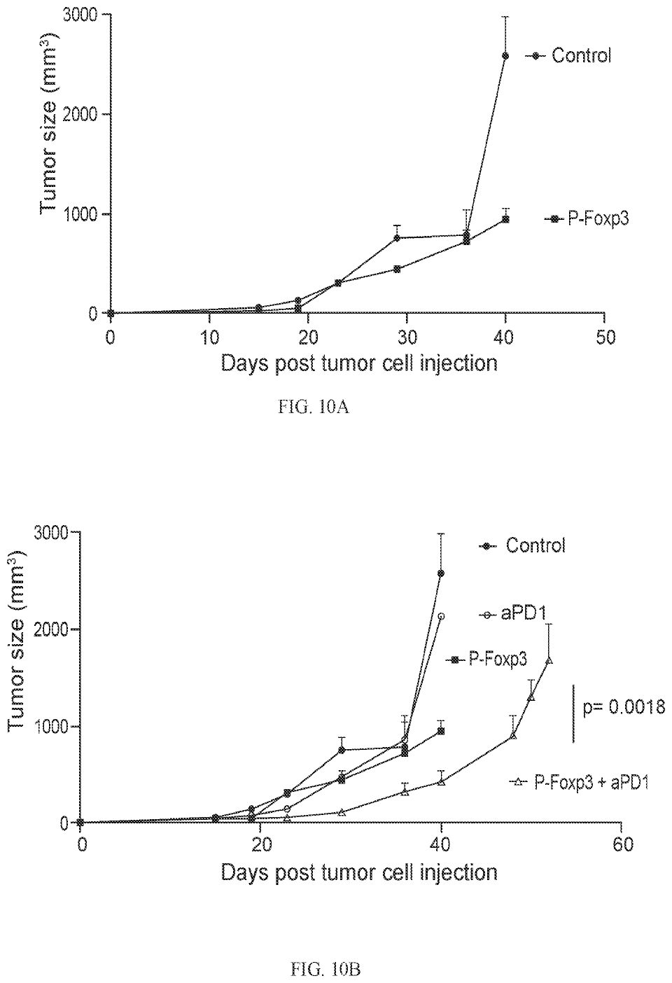

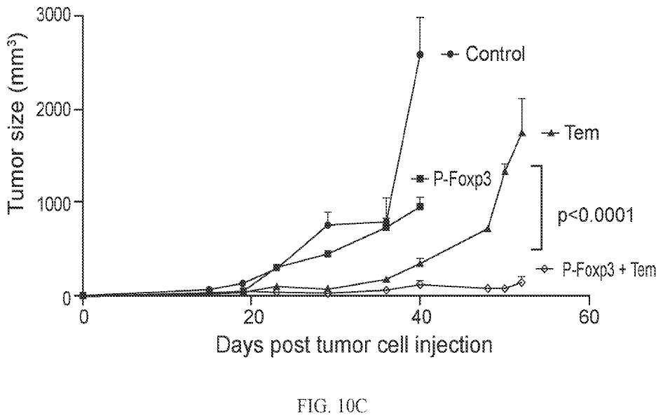

P-FoxP3 inhibited tumor growth in mice when combined with other immune modulating drugs such as an mTOR inhibitor (temsirolimus) or an αPD-1 antibody—herein using a monoclonal anti-mouse PD-1 antibody (BioXcell, BE0146) (FIGS. 10A-10C). P-FoxP3 alone appeared to decrease tumor growth compared to saline control (FIG. 3A), and the combination of P-FoxP3 and temsirolimus was better than temsirolimus alone in decreasing tumor growth (FIG. 3C). The combination of P-FoxP3 and αPD-1 antibody was better than αPD-1 antibody alone in decreasing tumor growth (FIG. 3B).

In comparison, P60 alone had no effect on RENCA tumor growth (FIG. 8A). The tumor in the P60 treated tumor grew at the same rate as the saline treated miceVarious embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of”.

1. A compound of chemical structure:
E3LBM-L-FOXP3BP,
wherein
the E3LBM is a ubiquitin ligase binding moiety;
the L is a bond or a chemical linker that is chemically linked to the E3LBM and the PFOX3M; and
the FOXP3BP is a peptide capable of binding to Forkhead box protein P3 (FOXP3), wherein upon binding of the FOXP3 to the compound, the FOXP3 is ubiquitinated by a ubiquitin ligase;
or a pharmaceutically acceptable salt, enantiomer, stereoisomer, solvate, polymorph, or prodrug thereof.

2. The compound of claim 1, wherein the FOXP3BP is a polypeptide comprising an amino acid sequence set forth in SEQ ID NO:1 or a variant thereof comprising an amino acid sequence that has at least 99%, 98%, 97%, 96%, 95%, 90%, 80%, or 60% sequence identity to SEQ ID NO:1.

3. The compound of claim 2, wherein the FOXP3BP is a polypeptide consisting of an amino acid sequence of SEQ ID NO:1.

4. The compound of claim 1, wherein the FOXP3BP is a polypeptide variant of SEQ ID NO:1, wherein one or more residues of SEQ ID NO:1 at positions 2, 3, 5, and/or 11 are substituted with L-alanine or a D-amino acid.

5. The compound of claim 2, wherein the E3LBM is selected from the group consisting of pomalidomide, thalidomide, and lenalidominde.

6. The compound of claim 1, wherein the E3LBM is a small molecule moiety that binds a ubiquitin ligase selected from the group consisting of cereblon, X-linked inhibitor of apoptosis (XIAP), von Hippel-Lindau (VHL), and mouse double minute 2 (MDM2).

7. The compound of claim 1, wherein the L is a polyethyleneglycol optionally substituted with aryl or phenyl, having from 1 to 10 ethylene glycol units.

8. A pharmaceutical composition comprising one or more compounds of claim 1, further comprising a pharmaceutically acceptable carrier, additive or excipient, and optionally further comprising an additional bioactive agent.

9. The pharmaceutical composition of claim 8, comprising the additional bioactive agent, which is selected from the group consisting of an mTOR inhibitor, a PD-1 inhibitor, a PD-L1 inhibitor, an immune checkpoint inhibitor, immunomodulating drugs, and a combination thereof.

10. The pharmaceutical composition of claim 9, wherein the mTOR inhibitor comprises temsirolimus: the PD-1 inhibitor comprises Pembrolizumab, Nivolumab, Dostarlimab, or Cemiplimab; the PD-L1 inhibitor comprises Atezolizumab, Avelumab, or Durvalumab: the immune checkpoint inhibitor comprises Relatlimab; and the immunomodulating drugs comprises Thalidomide, lenalidomide, and pomalidomide.

11-15. (canceled)

16. A method of inhibiting growth of a tumor in a subject, or treating the subject having a cancer, comprising administering to the subject a therapeutically effective amount of a compound according to claim 1.

17. The method of claim 16, wherein the E3LBM of the compound is derived from pomalidomide, the L of the compound is a polyethyleneglycol, and the FOXP3BP of the compound is a polypeptide having an amino acid sequence of SEQ ID NO:1 or a variant thereof.

18. The method of claim 16, further comprising administering to the subject a therapeutically effective amount of an mTOR inhibitor.

19. The method of claim 16, further comprising administering to the subject a therapeutically effective amount of a PD-1 inhibitor, a PD-L1 inhibitor, an immune checkpoint inhibitor, immunomodulating drugs, or a combination thereof.

20. (canceled)

21. The method of claim 18, wherein the administration comprises administering for about 2 weeks, 3 weeks, 4 weeks, or more, and the tumor in the subject is smaller in size, or the growth of the tumor as a percentage of initial size prior to the administration is smaller in the subject, compared to a subject having the cancer and treated without a combination of the compound and the mTOR inhibitor.

22. The method of claim 19, wherein the administration comprises administering for about 2 weeks, 3 weeks, 4 weeks, or more, and the tumor in the subject is smaller in size, or the growth of the tumor as a percentage of initial size prior to the administration is smaller in the subject, compared to a subject having the cancer and treated without a combination of the compound and the PD-1 inhibitor, a PD-L1 inhibitor, an immune checkpoint inhibitor, immunomodulating drugs, or a combination thereof.

23. A method of improving an anti-tumor effect of an immuno-oncology drug in a subject, comprising administering to the subject a therapeutically effective amount of a compound according to claim 1, wherein the subject has been treated or is in need of a treatment with the immuno-oncology drug.

24. The method of claim 23, wherein the immuno-oncology drug comprises an mTOR inhibitor, a PD-1 inhibitor, a PD-L1 inhibitor, an immune checkpoint inhibitor, immunomodulating drugs, or a combination thereof.

25. The method of claim 23, wherein the therapeutically effective amount of the compound is administered for about 2 weeks, 3 weeks, 4 weeks, or more.

26. The method of claim 23, wherein the compound is effective for reducing a number of CD4+ T cells that express the FOXP3 transcription factor in the subject.
